(19) United States
(12) Patent Application Publication
(10) Pub. No.: US 2020/0405657 A1
(43) Pub. Date: Dec. 31, 2020

(54) TREATMENT OF PAIN USING ALLOSTERIC MODULATOR OF TRPV1

(71) Applicant: GBS Global BioPharma, Inc., Ottawa (CA)

(72) Inventors: Andrea Small-Howard, Norwalk, CA (US); Helen Turner, Honolulu, HI (US)

(21) Appl. No.: 16/914,205

(22) Filed: Jun. 26, 2020

Related U.S. Application Data
(60) Provisional application No. 62/868,794, filed on Jun. 28, 2019.

(51) Int. Cl. A61K 31/01; A61K 9/51; A61K 36/185

(52) U.S. Cl. CPC A61K 31/01; A61K 36/185; A61K 9/5146

(57) ABSTRACT

Provided herein are methods of treating pain using pharmaceutical compositions that comprise an allosteric modulator of TRPV1, optionally in admixture with a TRPV1 ligand. The pharmaceutical composition is substantially free of THC and THCA. Also provided are methods of identifying an allosteric modulator of TRPV1 by analyzing binding to a specific binding pocket in TRPV1 and designing a complex mixture comprising the allosteric modulator for treating pain.

### CROSS-REFERENCE TO RELATED APPLICATIONS

This application claims the benefit of U.S. Provisional Application No. 62/868,794, filed Jun. 28, 2019, which is hereby incorporated in its entirety by reference.

### BACKGROUND

Pain is distressing, subjective, often debilitating and associated with autonomic responses such as sweating, tachycardia, hypertension and syncope. Pain may be chronic or acute, and is linked to restricted and decreased quality of daily life, opioid dependence, anxiety and depression, and poor perceived health. An estimated 20.4% (50 million) of U.S. adults have chronic pain and 8.0% of U.S. adults (19.6 million) have high-impact chronic pain with estimated economic costs of ˜$600 B/yr. Pain has a social and socioeconomic dimension. Both primary pain prevalence and the opioid addiction associated with it are drivers for the discovery of new analgesics which are efficacious and have low side-effect profiles. Opioid addiction affects 1.7M Americans, kills ˜50,000 individuals per year and has an emerging health economic burden of $78.5 B/yr. The Transient Receptor Superfamily (TRP) ion channel is known to be involved in certain types of pain, and is the molecular target for capsaicin-based topical analgesics.

There are major subtypes of neuropathic, nocioceptive and psychogenic pain. Neuropathy arises when somatosensory systems are damaged or diseased (e.g., chronic pain associated with diabetes, post-herpetic pain). Nocioception, from the Latin nocere (to harm, hurt) is a fundamental evolutionary adaptation to danger, injury or trauma. Sensory neurons respond to chemical (noxious small molecules), mechanical (pressure) and physical (heat, cold, osmolarity) changes in tissue and transmit signals via nerve fibers, the Dorsal Root Ganglia (DRG), and the spinal cord to the brain. Nocioceptive pain is thresholded, with the sensory neurons and the nocioceptive ion channels that control their activation having discrete and discontinuous activation barriers. For example, for heat sensitive nocioceptors, and the heat sensitive Transient Receptor Potential (TRP) family cation channels that gate their activation, thermosensation becomes pain at higher temperatures (45-52° C., TRPV1, TRPV2). Nocioceptive pain is a common event (and chronic health problem). Nocioceptive TRP channels are gated by a remarkably diverse set of potent small molecules that are encountered commonly (capsaicin, allicin, menthol, gingerol) as plant secondary metabolites. Their activations of TRP channels range from sensation to pain depending on dose and exposure. In additional, certain nocioceptive pathways are prone to hyperalgesia (chronic and abnormal sensitivity to pain) resulting from tissue damage-associated sensitization of sensory neurons.

Because of their role in nociception, TRP channels have been identified as targets for treating pain disorders. Both antagonism and agonism of the TRP channel have been exploited for pain management. For example, TRPV1 antagonists have utility in acute analgesia. For chronic pain management, TRPV1 agonists are typically used. This latter strategy exploits the fact that continued TRPV1 receptor agonism causes desensitization at the cell surface (receptor internalization, degradation and recycling). Prolonged agonism of TRPV1 also leads to calcium and sodium cationic overload of the TRPV1-containing sensory neuron, leading to cell death.

In practice, the use of TRPV1 agonists to effect desensitization involves topical application of high levels of a well-known TRPV1 agonist, capsaicin, repeatedly over time to the affected area. This therapeutic approach has the benefit of efficacy and low cost. However, it also has weaknesses.

First, high affinity and high specificity TRPV1 agonists target only TRPV1-containing nociceptors, leaving other sensory neurons and TRP channels involved in pain untouched. Second, use of high affinity and high specificity TRPV1 agonists such as capsaicin causes high levels of discomfort during initial treatment, in the period prior to desensitization. It is for this reason that post-herpetic pain is currently not addressable using TRPV1-mediated desensitization due to the highly irritant nature of the therapy on sensitive areas such as the gastric mucosa and reproductive tract mucosa. Third, capsaicin-mediated desensitization treatments are limited to topical use; visceral pain, headache and certain musculoskeletal pain disorders are not addressed by this therapy.

There is, therefore, a need for therapeutic TRPV1 ligands, such as TRPV1 agonists, that are more sophisticated than capsaicin and improve upon its mode of action, efficacy and side effect profiles. Such improved ligands should cause reduced pain during desensitization, thereby allowing topical treatment of sensitive body areas. There is a need for TRPV1 ligands with broader target specificity, able to target multiple types of TRP-bearing nociceptors, thereby improving the degree of tissue desensitization. There is also a need for TRPV1 ligands suitable for systemic administration in addition to topical application.

Such new medications would also be useful for the treatment of various diseases associated with TRPV1 other than pain. While TRP channels were first shown to be involved in pain and nociception, they are now known to have various other physiological roles, suggesting that they can be a target for treatment of other diseases. For example, TRP channels have been identified as a target for treatment of cardiovascular disease; targeted pharmacological inhibition of TRPV1 has been shown to significantly diminish cardiac hypertrophy in a mouse model. See U.S. Pat. No. 9,084,786. Chronic downregulation of TRPV1 levels by receptor desensitization with a TRPV1 agonist would therefore be expected to similarly protect, and potentially rescue, cardiac hypertrophy and its associated symptoms and outcomes (cardiac remodeling, cardiac fibrosis, apoptosis, hypertension, or heart failure). However, there is currently no TRPV1 agonist suitable for systemic administration and suitable for chronic downregulation of TRPV1 in a visceral organ, and there is therefore a need to develop such approaches in an analogous manner to the chronic pain approaches described above.

Thus, there exists a need to find new methods for TRPV1 regulation. Such new compounds or pharmacological compositions would provide novel and more effective ways of treating various diseases associated with the TRPV1 channel, and potentially other members of the TRPV1 family, including pain disorders and cardiovascular diseases.

### SUMMARY

Cannabis has been used for millennia to provide analgesia and treat various types of pain. However, the use of whole plant C. sativa extracts obtained from dispensaries as ‘medicine’ is beset by issues of psychoactive adverse effects (due to the presence of delta9-tetrahydracannabinol, THC), lack of consistency and standardization, contamination (microbial, pesticide), and inadequate evidence of efficacy. All of these place patients at risk and limit the potential utility of Cannabis-derived compounds as therapeutics. There are unmet needs to evaluate the major components of the Cannabis secondary metabolome (several hundred cannabinoids and terpenes), discriminate active therapeutic from inactive or dispensable compounds, and reformulate single compounds or mixtures for prescription using accepted regulatory pathways.

In the earlier studies described in U.S. application Ser. No. 15/986,316 and PCT/US2018/033956, which are incorporated by reference in their entireties herein, we demonstrated that Cannabis exerts its anti-nociceptive effects at least in part through the TRPV1 receptor, with both cannabinoids and terpenes acting as agonists. We further demonstrated that Myrcene contributes significantly to the observed TRPV1 agonism, and that like Capsaicin, causes TRPV1 desensitization after prolonged exposure. The earlier studies, however, could not identify TRPV1 agonism by other components of C. sativa extracts (e.g., terpenes other than myrcene, cannabinoids), probably due to limitations of in vitro screening methods used in the study.

In the present disclosure, we have identified additional components of C. sativa extracts that have TRPV1 agonism and exert anti-nociceptive effects by using a different strategy, i.e., identifying compounds having chemical moieties predicted to bind to a specific binding pocket of TRPV1. This strategy was based on the discovery of the binding pockets of TRPV1 specific to Myrcene (“site 4”) or Cannabidiol (CBD) (“site 4A”), in particular, key amino acid residues implicated in binding and acutely activating the TRPV1 channel without causing the state transition of TRPV1 to a dilated state and the calculation of the relative binding energies of compounds for these sites. The use of newly discovered binding sites for screening of the target compounds allowed pre-screening of numerous and structurally diverse terpenes and cannabinoids and identification of those that have the desired modulatory effects similar to myrcene or cannabidiol (CBD), such as acute activation, long term desensitization and allosteric modulation of TRPV1 without the state transition. Terpene and cannabinoid compounds binding to the binding pockets 4 and 4A are believed to have desirable pharmaceutical properties relative to Capsaicin. Binding pockets 4 and 4A contain some residues that are implicated in binding to Capsaicin, but also implicate multiple residues and a three-dimensional pocket conformation that is discrete from known mechanisms of Capsaicin binding. The combination of molecular modeling and functional data that we have support the idea that ligands fully interacting with sites 4 or 4A can act allosterically with other ligands and maintain TRPV1 channel in a specific non-dilated state without transition to dilated state, possibly via effects on the S4-S5 linker that are discrete from those caused by Capsaicin.

Additionally, to assess the broader therapeutic potential of Myrcene or other compounds binding to the same or overlapping binding pockets, we generated a target analysis and disease-prediction network for myrcene and nerolidol using a proprietary in silico prediction approach, termed the GB Sciences' Network Pharmacology Platform (“NPP”). This in silico prediction method can extend the likely indications of compounds that bind pockets 4 or 4A to previously unrecognized disease processes that have been linked to the compound and TRPV1 or similar channels through the compound-to-gene-to-disease approach used in the NPP.

Accordingly, in a first aspect, the present disclosure provides a method of designing complex multi-component mixtures (pharmaceutical compositions) of compounds to treat pain through targeting the TRPV1 or similar ion channel such as TRPV2, TRPM8, and TRPA1. The method comprising: a process where binding of compounds to specific interaction sites (such as site 4 or 4A identified below and future sites to be identified using the methodology demonstrated here), either demonstrated in vitro or predicted using in silico techniques, is used to differentiate between likely analgesic and non-analgesic efficacy of compounds occurring in Cannabis or other plants at the ion channels indicated, allowing for the rational and informed design of novel complex compound mixtures that target pain. For example, (i) in discriminating between desirable, dispensable, or neutral TRPV1-targeting terpenes for the design of Cannabis-derived mixtures for analgesia, the presence of the functional dimethyl moiety is used as a molecular discriminator of likely binding to site 4 (identified below), leading to the inclusion of compounds (including but not limited to myrcene, ocimene, linalool, nerolidiol, bisabolol) that contain the moiety but the exclusion of compounds (including but not limited to caryophyllene, camphene, pinene) that do not contain the moiety in rational mixture design for analgesia. (ii) in designing the structural features of novel synthetic compounds to be included in rationally-designed TRPV1-targeting complex compound mixtures for analgesia.

In another aspect, the present disclosure provides a method of treating pain in a mammalian subject, the method comprising: administering to the subject a pharmaceutical composition, in an amount, by a route of administration, and for a time sufficient to cause TRPV1 inactivation or desensitization in sensory neurons within the subject, wherein the pharmaceutical composition comprises single compounds or multi-component mixtures of (i) a Cannabis-derived compound that binds to TRPV1 in a pocket comprised of site 4 or 4A (identified below), and activates the channel for Ca²⁺ and sodium permeation but may or may not initiate pore dilation and state transition depending upon the desired therapeutic outcome, or (ii) a synthetic compound designed to bind to TRPV1 in a pocket comprised of site 4 or 4A, that activates the channel but may or may not initiate pore dilation and state transition, or (iii) a naturally-occurring compound that binds to TRPV1 in a pocket comprised of site 4 or 4A, and activates the channel but may or may not initiate pore dilation and state transition, (iv) either (i), (ii) or (iii) with a pharmaceutically acceptable carrier or diluent.

In yet another aspect, the present disclosure provides a method of treating pain in a mammalian subject, the method comprising: administering to the subject a pharmaceutical composition, in an amount, by a route of administration, and for a time sufficient to allosterically modulate TRPV1 activation status wherein the pharmaceutical composition comprises an allosteric modulator and one other TRPV1 ligand where the allosteric modulator is either (i) Myrcene or (ii) a Cannabis-derived compound that binds to TRPV1 in a pocket comprised of site 4, or (iii) a synthetic compound designed to bind to TRPV1 in a pocket comprised of site 4, or (iv) a naturally-occurring compound that binds to TRPV1 in a pocket comprised of site 4, and (v) either (i), (ii), (iii) or (iv) with a pharmaceutically acceptable carrier or diluent; and where the other TRPV1 ligand is a Cannabis-derived, synthetic or other plant/natural source-derived compound that binds TRPV1 in a manner overlapping or partially overlapping with site 4.

In one aspect, the present disclosure provides a method of designing a complex mixture for treating pain through targeting a TRP channel selected from TRPV1, TRPV2, TRPM8 and TRPA1, comprising the steps of: analyzing compounds in Cannabis or other plants using in vitro or in silico technique and predicting whether each of the compounds binds to site 4 or site 4A of TRPV1, thereby differentiating between likely analgesic and non-analgesic compounds; selecting a subset of the compounds that contain a functional dimethyl moiety and excluding a different subset of the compounds that do not contain the functional dimethyl moiety, thereby obtaining selected compounds; and designing the complex mixture comprising the selected compounds.

In some embodiments, the site 4 of TRPV1 is a binding pocket of a set of amino acid residues, wherein the amino acid residues comprise: Arg 491, Asn 437, Phe 434, Tyr 555, Ser 512, Tyr 554, Glu 513, Phe 516, and Phe 488 of rat TRPV1 or the closely equivalent human TRPV1 residues. In some embodiments, the site 4A of TRPV1 is a binding pocket of a set of amino acid residues, wherein the amino acid residues comprise: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues. In some embodiments, the method further comprises the step of identifying compounds that do not initiate state transition or pore dilation in TRPV1.

In a different aspect, the present disclosure provides a method of treating pain in a mammalian subject, comprising the steps of: administering to the subject a pharmaceutical composition, in an amount, by a route of administration, and for a time sufficient to cause TRPV1 inactivation or desensitization in sensory neurons within the subject, wherein the pharmaceutical composition comprises an active compound capable of activating TRPV1 by binding to site 4 or 4A of TRPV1, and a pharmaceutically acceptable carrier or diluent; and wherein the active compound is (i) a naturally occurring compound, optionally a Cannabis-derived compound, or (ii) a synthetic compound.

In some embodiments, the active compound does not initiate TRPV1 pore dilation and state transition. In some embodiments, the site 4 of TRPV1 is a binding pocket of a set of amino acid residues, wherein the amino acid residues comprise: Arg 491, Asn 437, Phe 434, Tyr 555, Ser 512, Tyr 554, Glu 513, Phe 516, and Phe 488 of rat TRPV1 or the closely equivalent human TRPV1 residues. In some embodiments, the site 4A of TRPV1 is a binding pocket of a set of amino acid residues, wherein the amino acid residues comprise: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues.

In some embodiments, the active compound is selected from the group consisting of β-ocimene, linalool, nerolidol, and bisabolol. In some embodiments, the active compound is Myrcene. In some embodiments, the active compound is not Myrcene. In some embodiments, the active compound is Cannabidiol (CBD).

In some embodiments, the pharmaceutical composition further comprises a PLGA nanoparticle. In some embodiments, the PLGA nanoparticle comprises PLGA copolymer having a ratio of lactic acid to glycolic acid between about 10-90% lactic acid and about 90-10% glycolic acid. In some embodiments, the ratio of lactic acid to glycolic acid is about 10% lactic acid to about 90% glycolic acid. In some embodiments, the ratio of lactic acid to glycolic acid is about 25% lactic acid to about 75% glycolic acid. In some embodiments, the ratio of lactic acid to glycolic acid is about 50% lactic acid to about 50% glycolic acid. In some embodiments, the ratio of lactic acid to glycolic acid is about 75% lactic acid to about 25% glycolic acid. In some embodiments, the ratio of lactic acid to glycolic acid is about 90% lactic acid to about 10% glycolic acid.

In some embodiments, the active compound is present in the pharmaceutical composition in an amount that is at least 10% (w/w) of the total content of terpenes and cannabinoids. In some embodiments, the active compound is present in an amount that is at least 25% (w/w) of the total content of terpenes and cannabinoids. In some embodiments, the active compound is present in an amount that is at least 50% (w/w) of the total content of terpenes and cannabinoids. In some embodiments, the active compound is present in an amount that is at least 75% (w/w) of the total content of terpenes and cannabinoids. In some embodiments, the active compound is present in an amount that is at least 90% (w/w) of the total content of terpenes and cannabinoids.

In some embodiments, the sensory neurons are nociceptive neurons. In some embodiments, the sensory neurons are peripheral nociceptive neurons. In some embodiments, the sensory neurons are visceral nociceptive neurons. In some embodiments, the pain is neuropathic pain. In some embodiments, the pain is diabetic peripheral neuropathic pain. In some embodiments, the pain is post-herpetic neuralgia.

In some embodiments, the pharmaceutical composition is administered at least once a day for more than 7 days. In some embodiments, the pharmaceutical composition is administered at a dose, by a route of administration, and on a schedule sufficient to maintain effective levels of the active compound at the sensory neuron nociceptors for at least 3 days. In some embodiments, the pharmaceutical composition is administered at a dose, by a route of administration, and on a schedule sufficient to maintain effective levels of the active compound at the sensory neuron nociceptors for at least 7 days. In some embodiments, the pharmaceutical composition is administered topically, systemically, intravenously, subcutaneously, or by inhalation.

In yet another aspect, the present disclosure provides a method of treating pain in a mammalian subject, comprising the steps of: administering to the subject a pharmaceutical composition, in an amount, by a route of administration, and for a time sufficient to cause TRPV1 inactivation or desensitization in sensory neurons within the subject, wherein the pharmaceutical composition comprises (i) an allosteric modulator capable of activating TRPV1 by binding to site 4 of TRPV1, (ii) a TRPV1 ligand capable of activating TRPV1 by binding to a ligand-binding site at least partially overlapping with the site 4 of TRPV1, and (iii) a pharmaceutically acceptable carrier or diluent, wherein the allosteric modulator and the TRPV1 ligand is naturally occurring, optionally Cannabis-derived, or synthesized; and wherein the allosteric modulator and the TRPV1 ligand are different compounds.

In some embodiments, the allosteric modulator is Myrcene. In some embodiments, the allosteric modulator is not Myrcene. In some embodiments, the allosteric modulator is selected from the group consisting of β-ocimene, linalool, nerolidol, and bisabolol.

In some embodiments, the TRPV1 ligand is cannabidiol (CBD). In some embodiments, the ligand-binding site is site 4A.

In some embodiments, the site 4 of TRPV1 is a binding pocket of a set of amino acid residues, wherein the amino acid residues comprise: Arg 491, Asn 437, Phe 434, Tyr 555, Ser 512, Tyr 554, Glu 513, Phe 516, and Phe 488 of rat TRPV1 or the closely equivalent human TRPV1 residues. In some embodiments, the site 4A of TRPV1 is a binding pocket of a set of amino acid residues, wherein the amino acid residues comprise: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues (see Table 2).

In some embodiments, the allosteric modulator or the TRPV1 ligand does not initiate TRPV1 dilation and state transition.

In some embodiments, the pharmaceutical composition further comprises a PLGA nanoparticle. In some embodiments, the PLGA nanoparticle comprises PLGA copolymer having a ratio of lactic acid to glycolic acid between about 10-90% lactic acid and about 90-10% glycolic acid. In some embodiments, the ratio of lactic acid to glycolic acid is about 10% lactic acid to about 90% glycolic acid. In some embodiments, the ratio of lactic acid to glycolic acid is about 25% lactic acid to about 75% glycolic acid. In some embodiments, the ratio of lactic acid to glycolic acid is about 50% lactic acid to about 50% glycolic acid. In some embodiments, the ratio of lactic acid to glycolic acid is about 75% lactic acid to about 25% glycolic acid. In some embodiments, the ratio of lactic acid to glycolic acid is about 90% lactic acid to about 10% glycolic acid.

In some embodiments, the allosteric modulator and the TRPV1 ligand are present in the pharmaceutical composition in an amount that is at least 10% (w/w) of the total content of terpenes and cannabinoids. In some embodiments, the allosteric modulator and the TRPV1 ligand are present in an amount that is at least 25% (w/w) of the total content of terpenes and cannabinoids. In some embodiments, the allosteric modulator and the TRPV1 ligand are present in an amount that is at least 50% (w/w) of the total content of terpenes and cannabinoids. In some embodiments, the allosteric modulator and the TRPV1 ligand are present in an amount that is at least 75% (w/w) of the total content of terpenes and cannabinoids. In some embodiments, the allosteric modulator and the TRPV1 ligand are present in an amount that is at least 90% (w/w) of the total content of terpenes and cannabinoids.

In some embodiments, the sensory neurons are nociceptive neurons. In some embodiments, the sensory neurons are peripheral nociceptive neurons. In some embodiments, the sensory neurons are visceral nociceptive neurons. In some embodiments, the pain is neuropathic pain. In some embodiments, the pain is diabetic peripheral neuropathic pain. In some embodiments, the pain is post-herpetic neuralgia.

In some embodiments, the pharmaceutical composition is administered at least once a day for more than 7 days. In some embodiments, the pharmaceutical composition is administered at a dose, by a route of administration, and on a schedule sufficient to maintain effective levels of the allosteric modulator or the TRPV1 ligand at the sensory neuron nociceptors for at least 3 days. In some embodiments, the pharmaceutical composition is administered at a dose, by a route of administration, and on a schedule sufficient to maintain effective levels of the allosteric modulator or the TRPV1 ligand at the sensory neuron nociceptors for at least 7 days. In some embodiments, the pharmaceutical composition is administered topically, systemically, intravenously, subcutaneously, or by inhalation.

In one aspect, the present invention provides a pharmaceutical composition, comprising: an allosteric modulator capable of activating TRPV1 by binding to site 4 of TRPV1 and a pharmaceutically acceptable carrier or diluent, wherein the composition is substantially free from THC; and wherein the allosteric modulator is a naturally occurring compound, optionally Cannabis-derived compound, or synthesized compound.

In some embodiments, the site 4 of TRPV1 is a binding pocket of a set of amino acid residues, wherein the amino acid residues comprise: Arg 491, Asn 437, Phe 434, Tyr 555, Ser 512, Tyr 554, Glu 513, Phe 516, and Phe 488 of rat TRPV1 or the closely equivalent human TRPV1 residues.

In some embodiments, the pharmaceutical composition further comprises a TRPV1 ligand capable of activating TRPV1 by binding to a ligand-binding site at least partially overlapping with the site 4 of TRPV1, wherein the TRPV1 ligand is a naturally occurring compound, optionally Cannabis-derived compound, or synthesized compound.

In some embodiments, the ligand-binding site is site 4A. In some embodiments, the site 4A of TRPV1 is a binding pocket of a set of amino acid residues, wherein the amino acid residues comprise: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues. In some embodiments, the allosteric modulator or the TRPV1 ligand does not initiate TRPV1 dilation and state transition.

In some embodiments, the allosteric modulator is Myrcene. In some embodiments, the allosteric modulator is not Myrcene. In some embodiments, the allosteric modulator is selected from the group consisting of β-ocimene, linalool, nerolidol, and bisabolol. In some embodiments, the TRPV1 ligand is cannabidiol (CBD).

In some embodiments, the pharmaceutical composition comprises no terpene other than the allosteric modulator. In some embodiments, the pharmaceutical composition comprises no terpene that binds to the site 4, other than the allosteric modulator.

In some embodiments, the pharmaceutical composition comprises a PLGA nanoparticle. In some embodiments, the PLGA nanoparticle comprises PLGA copolymer having a ratio of lactic acid to glycolic acid between about 10-90% lactic acid and about 90-10% glycolic acid. In some embodiments, the ratio of lactic acid to glycolic acid is about 10% lactic acid to about 90% glycolic acid. In some embodiments, the ratio of lactic acid to glycolic acid is about 25% lactic acid to about 75% glycolic acid. In some embodiments, the ratio of lactic acid to glycolic acid is about 50% lactic acid to about 50% glycolic acid. In some embodiments, the ratio of lactic acid to glycolic acid is about 75% lactic acid to about 25% glycolic acid. In some embodiments, the ratio of lactic acid to glycolic acid is about 90% lactic acid to about 10% glycolic acid.

In some embodiments, the allosteric modulator and the TRPV1 ligand are present in an amount that is at least 10% (w/w) of the total content of terpenes and cannabinoids. In some embodiments, the allosteric modulator and the TRPV1 ligand are present in an amount that is at least 25% (w/w) of the total content of terpenes and cannabinoids. In some embodiments, the allosteric modulator and the TRPV1 ligand are present in an amount that is at least 50% (w/w) of the total content of terpenes and cannabinoids. In some embodiments, the allosteric modulator and the TRPV1 ligand are present in an amount that is at least 75% (w/w) of the total content of terpenes and cannabinoids. In some embodiments, the allosteric modulator and the TRPV1 ligand are present in an amount that is at least 90% (w/w) of the total content of terpenes and cannabinoids.

In some embodiments, the composition is formulated for topical, oral, buccal, sublingual, intravenous, intramuscular, subcutaneous, or inhalation administration. In some embodiments, the composition is formulated for administration by vaporizer, nebulizer, or aerosolizer. In some embodiments, the composition is lyophilized.

These and other aspects of the invention are described in further detail below.

### BRIEF DESCRIPTION OF THE DRAWINGS

FIG. 1 illustrates that the inducible expression of TRPV1 in a non-TRPV1 containing cell type confers capsaicin-sensitive calcium flux responses upon the cells. Here, HEK cells transfected with a rat TRPV1 gene under the control of a tetracycline-inducible promoter were induced to transcribe the TRPV1 gene and synthesize TRPV1 protein through the application of tetracycline for 16 h at 1 micromolar. This establishes that the experimental system used in the following studies clearly, and specifically, reports TRPV1-specific calcium fluxes, since capsaicin is a specific ligand for TRPV1.

FIGS. 2A-2C illustrate that terpenes contribute significantly to calcium fluxes via TRPV1 induced by Cannabis-derived mixtures of cannabinoids and terpenes. FIG. 2A shows calcium influx (relative fluorescence unit, “Fluo-4 RFU”) over time (in seconds, “s”) in HEK cells transfected with a construct encoding TRPV1, first without stimulus (“NS”), then after application of vehicle (“veh”), and after application of Strain A Mixture (“Strain mixture”). FIG. 2B shows calcium influx in TRPV1-expressing HEK cells after application of a mixture that includes only the cannabinoids present in the Strain A Mixture (“Cannabinoid Mixture”). FIG. 2C shows calcium influx in TRPV1-expressing HEK cells after application of a mixture that includes only the terpenes present in the Strain A Mixture (“Terpene Mixture”).

FIGS. 3A-3L illustrate that individual terpenes differentially contribute to calcium fluxes induced by the Terpene Mixture via TRPV1. FIG. 3A presents calcium influx over time in HEK cells transfected with a construct encoding TRPV1 without stimulus (“NS”), after application of vehicle (“veh”), and after application of the Terpene Mixture (“all terpenes”). FIGS. 3B-3L graph baseline-subtracted calcium influx over time in the TRPV1-expressing HEK cells separately for each of the terpenes present in the Terpene Mixture used in FIG. 3A—specifically, FIG. 3B for caryophyllene, FIG. 3C for limonene, FIG. 3D for myrcene, FIG. 3E for linalool, FIG. 3F for alpha bisabolol, FIG. 3G for humulene, FIG. 3H for beta pinene, FIG. 3I for nerolidol, FIG. 3J for camphene, FIG. 3K for ocimene, and FIG. 3L for alpha pinene.

FIG. 4 illustrates that myrcene contributes significantly to TRPV1-mediated calcium responses seen with the Terpene Mixture (“Terpenes”), but does not constitute 100% of the signal. Data were obtained from HEK cells transfected with and inducibly expressing TRPV1.

FIGS. 5A-5C illustrate that the measured calcium responses depend wholly or in part on the presence of the TRPV1 ion channel. FIG. 5A shows calcium influx over time in HEK wild type cells (“HEK wild type”) and in HEK cells transfected with and induced to express TRPV1 through the application of tetracycline (1 μM for 16 hours) (“HEK+TRPV1”) after application of the complete Strain A mixture. FIG. 5B shows calcium influx over time in HEK wild type cells and in HEK+TRPV1 cells after application of a mixture that includes only the cannabinoids present in the Strain A Mixture (“Cannabinoid Mixture”). FIG. 5C shows calcium influx over time in HEK wild type cells and in HEK+TRPV1 cells after application of a mixture that includes only the terpenes present in the Strain A Mixture (“Terpene Mixture”). All data are vehicle subtracted.

FIGS. 6A-6D illustrate that the myrcene-induced calcium influx depends wholly or in part on the presence of the TRPV1 ion channel. FIGS. 6A-6B show calcium influx over time in HEK wild type cells (“HEK wild type”) and in HEK+TRPV1 cells after application of myrcene at various concentrations: 3.5 μg/mg (FIG. 6A), 1.75 μg/mg (FIG. 6B), 0.875 μg/mg (FIG. 6C), and 0.43 μg/mg (FIG. 6D). All data are baseline subtracted.

FIGS. 7A-7B illustrate that the measured myrcene-induced calcium influx responses are inhibited by a specific pharmacological inhibitor of the TRPV1 ion channel. FIG. 7A shows calcium influx in TRPV1-expressing HEK cells over time (in seconds) in response to application of vehicle (“veh”), myrcene at 3.5 μg/ml, and further addition of the TRPV1 inhibitor, capsazepine (10 μM). FIG. 7B shows calcium influx in TRPV1-transfected HEK cells over time (in seconds) in response to application of vehicle (“veh”), myrcene at 3.5 μg/ml, and further addition of phosphate-buffered saline (“PBS”) instead of capsazepine. Data are baseline-subtracted.

FIGS. 8A-8D illustrate that when myrcene is applied in the absence of external calcium, at high concentrations it can induce TRPV1-dependent calcium release from internal stores. FIGS. 8A-8D present cytosolic calcium influxes over time in transfected HEK cells expressing TRPV1 (“HEK TRPV1”) or a wild-type HEK cells (“HEK wild type”) in response to various concentrations of myrcene—3.5 μg/ml (FIG. 8A), 1.75 μg/ml (FIG. 8B), 0.875 μg/ml (FIG. 8C) and 0.43 μg/ml (FIG. 8D) of myrcene. Experiments were conducted in the absence of external calcium in the medium. All data are baseline subtracted.

FIGS. 9A-9G illustrate that cannabinoids differentially contribute to calcium fluxes via TRPV1. FIGS. 9A-9G show calcium influx over time (seconds, “sec”) in HEK wild type cells and HEK cells expressing TRPV1 individually for each of the cannabinoids present in the Cannabinoid Mixture tested in FIG. 2B, specifically, FIG. 9A for cannabidivarin, FIG. 9B for cannabidigerol, FIG. 9C for cannabichromene, FIG. 9D for cannabigerolic acid, FIG. 9E for cannabidiol, FIG. 9F for cannabinol, and FIG. 9G for cannabidiolic acid. All stimuli were added at 20 seconds. All data are baseline subtracted.

FIG. 10 illustrates that Therapeutic Target Data Base (“TTD”) Enrichment Analysis tends to prioritize Myrcene over Nerolidol for development in pain and cardiovascular areas. In addition, myrcene contributes significantly to the predicted disease target set for native Cannabis.

FIG. 11 illustrates that diverse ion channel targets are predicted for direct or indirect modulation by myrcene.

FIG. 12 illustrates that limited ion channel targets or CNS-active targets are predicted for direct or indirect modulation by nerolidol.

FIG. 13 provides a table summarizing Imax measured over multiple sequential applications of cannabidiol (CBD), myrcene (MYR) or capsaicin (CAP), illustrating that each of these ligands can cause desensitization but in a different manner, which provides the potential for sophisticated control of the channel properties in analgesia.

FIG. 14 lists the compounds used in the experiments described.

FIG. 15 show a target analysis and disease-prediction network for one terpene, myrcene. The data were generated in silico using GB Sciences' Network Pharmacology Platform (“NPP”). The presence of multiple TRP channels in the network indicates that efficacy of myrcene will likely extend beyond TRPV1 to other nociceptive neurons in which the primary pain-conducting channel is a distinct TRP.

FIG. 16 show a target analysis and disease-prediction network for one terpene, nerolidol. The data were generated in silico using GB Sciences' NPP. The presence of multiple TRP channels in the network indicates that efficacy of myrcene will likely extend beyond TRPV1 to other nociceptive neurons in which the primary pain-conducting channel is a distinct TRP.

FIG. 17 illustrates the desirability for effective analgesics to target multiple ionotropic TRP receptors in the nociceptive nerve bundle.

FIGS. 18A-18C illustrate TRPV1 ion channel activation in single HEK293 cells overexpressing TRPV1 after application of increasing amounts of myrcene (M). FIG. 18A shows 5 μM myrcene, FIG. 18B shows 10 μM myrcene, and FIG. 18C shows 150 μM myrcene.

FIGS. 19A-19E illustrate electrophysiology data in single HEK293 cells overexpressing TRPV1 after addition of 5 μM myrcene (M) and 1 μM capsaicin (Cap). FIG. 19A shows the inward and outward ion current (nA) of the cell before and after myrcene and capsaicin addition. FIG. 19B is an enlarged view of FIG. 19A to show the myrcene-induced response. FIGS. 19C-19E show the I-V curve of the cell before application of myrcene or capsaicin (FIG. 19C), or after application of 5 μM myrcene (FIG. 19D) or 1 μM capsaicin (FIG. 19E).

FIG. 20A shows the current induced by application of 30 μM CBD in cells expressing TRPV1, and FIGS. 20B-20C show reduction of the current by application of capsazepine (FIG. 20B) and washout of CBD (FIG. 20C). FIG. 20D shows rectifying current with Eᵣₑᵥ of ˜0 mV (FIG. 20D).

FIG. 21 illustrates the responses when Myrcene is allowed to occupy the channel initially with 0 mM external Ca²⁺ concentration (i), which prohibited Ca²⁺ influx. When Ca²⁺ concentration was then introduced with 1 mM external Ca²⁺ (ii and iii), the response of Cannabidiol, as a second stimulus, is suppressed compared to without prior Myrcene incubation.

FIGS. 22A-22B illustrate the molecular docking of Myrcene at TRPV1 binding site #4.

FIG. 23 illustrates a method and research process in which molecular docking of a specific terpene or other compound identifies a site in the ionotropic TRP receptor, and once the implicated residues and their relationship to the structure of the ligand are known, as well as their relative binding energies, that desirable moieties can be identified. These moieties can be used to then discriminate between naturally occurring ligands in silico, or incorporated into the rational design process for synthetic ligands. For example, this figure illustrates the process of differentiating between groups of Cannabis terpenes that share the common moiety and may have the capacity and affinity to occupy binding site #4 of TRPV1 (Site 4 Type Terpenes), as well as terpenes that are unlikely to occupy site #4 of TRPV1 (Non-site 4 Type Terpenes).

FIG. 24 shows the molecular docking of Cannabidiol (CBD) at a binding site #4A of TRPV1.

FIG. 25A illustrates an alternative representation of the molecular docking of Cannabidiol (CBD) at the same binding site of TRPV1 as FIG. 24. FIG. 25B provides an enlarged image of FIG. 25A.

FIG. 26 illustrates the implicated residues in the binding of Myrcene across a two-dimensional representation of the channel's protein sequence.

FIG. 27 illustrates the implicated residues in the binding of CBD across a two-dimensional representation of the channel's protein sequence.

### DETAILED DESCRIPTION

### Definitions

Unless defined otherwise, all technical and scientific terms used herein have the meaning commonly understood by a person skilled in the art to which this invention belongs. As used herein, the following terms have the meanings ascribed to them below.

“Myrcene” (synonymously “β-myrcene”) is 7-methyl-3-methylideneocta-1,6-diene and illustrated by the structural formula

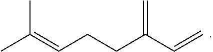

“α-Ocimene” is cis-3,7-dimethyl-1,3,7-octatriene and illustrated by the structural formula

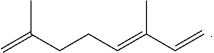

“cis-β-Ocimene” is (Z)-3,7-dimethyl-1,3,6-octatriene and illustrated by the structural formula

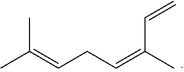

“trans-β-Ocimene” is (E)-3,7-dimethyl-1,3,6-octatriene and illustrated by the structural formula

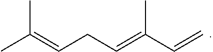

“Linalool” is 3,7-dimethyl-1,6-octadien-3-ol and illustrated by the structural formula

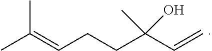

“Nerolidol” is 3,7,11-Trimethyl-1,6,10-dodecatrien-3-ol and illustrated by the structural formula

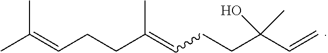 [SMILES: C=CC(C)(O)CCC=C(C)CCC=C(C)C]

“cis-nerolidol” is the cis-isomer of nerolidol and illustrated by the structural formula

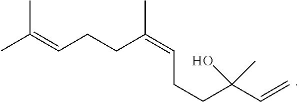 [SMILES: C=CC(C)(O)CC/C=C(/C)CCC=C(C)C]

“trans-nerolidol” is the trans-isomer of nerolidol and illustrated by the structural formula

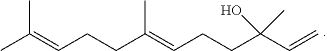 [SMILES: C=CC(C)(O)CC/C=C(\C)CCC=C(C)C]

“Bisabolol” is 6-methyl-2-(4-methylcyclohex-3-en-1-yl)hept-5-en-2-ol and illustrated by the structural formula

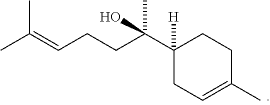 [SMILES: CC(C)=CCC[C@@](C)(O)[C@@H]1CC=C(C)CC1]

“Dimethylallyl” group refers to an unsaturated C₅H9 alkyl substituent as illustrated by the formula

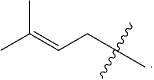 [SMILES: *CC=C(C)C]

“Dimethylallyl” group can be a functional dimethyl moiety.

“Functional dimethyl moiety” as used herein refers to a moiety comprising a dimethyl group that can bind to TRPV1.

“Terpene” means one of the compound selected from the group consisting of alpha-bisabolol (α-bisabolol), alpha-humulene (α-humulene), alpha-pinene (α-pinene), beta-caryophyllene (β-caryophyllene), myrcene, (+)-beta-pinene (β-pinene), camphene, limonene, linalool, phytol, and nerolidol.

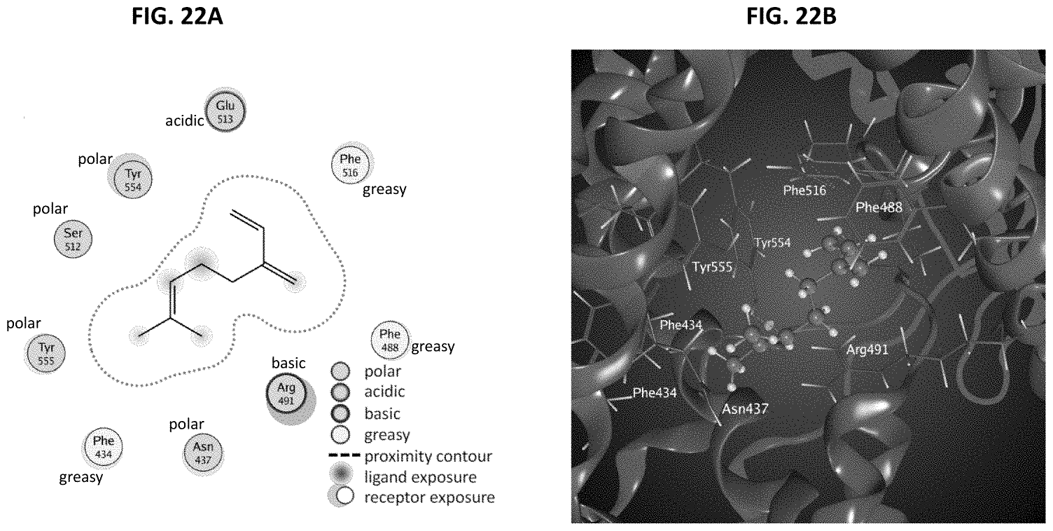

“Site 4” as used herein refers to a binding site of myrcene in TRPV1, as depicted in FIG. 22A and FIG. 22B. Site 4 can be a binding pocket of a set of amino acid residues in TRPV1 comprising at least two, three, four, five, six, seven, eight, or nine amino acid residues selected from the group consisting of: Arg 491, Asn 437, Phe 434, Tyr 555, Ser 512, Tyr 554, Glu 513, Phe 516, and Phe 488 of rat TRPV1 or the closely equivalent human TRPV1 residues (see Table 2). In preferred embodiments, Site 4 is a binding pocket of a set of amino acid residues comprising Arg 491, Asn 437, Phe 434, Tyr 555, Ser 512, Tyr 554, Glu 513, Phe 516, and Phe 488 of rat TRPV1 or the closely equivalent human TRPV1 residues (see Table 2).

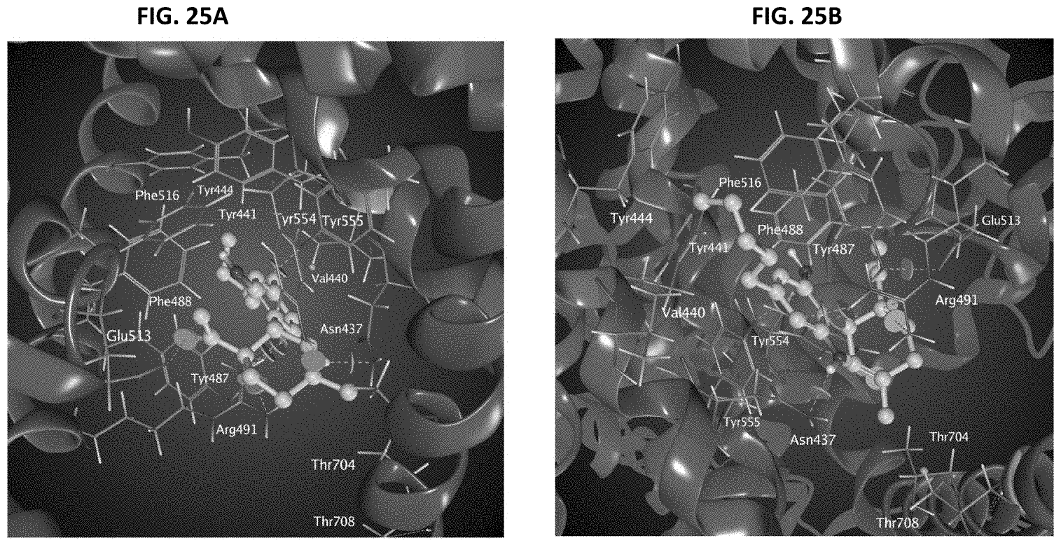

“Site 4A” as used herein refers to a binding site of cannabidiol (CBD) in TRPV1, as depicted in FIG. 25A and FIG. 25B. Site 4A can be a binding pocket of a set of amino acid residues in TRPV1 comprising at least two, three, four, five, six, seven, eight, nine, ten, eleven, twelve, thirteen, or fourteen amino acid residues selected from the group consisting of: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues (see Table 2). In preferred embodiments, site 4 is a binding pocket of a set of amino acid residues in TRPV1 comprising Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues (see Table 2).

An “allosteric modulator of TRPV1” refers to a compound that can bind to Site 4 of TRPV1 and allosterically modulate activity of TRPV1. In some embodiments, an allosteric modulator of TRPV1 is a terpene having a dimethyl moiety but is not limited to terpenes.

A “TRPV1 ligand” refers to a compound that can bind to Site 4A of TRPV1 and activate TRPV1. In preferred embodiments, a TRPV1 ligand can keep TRPV1 in a non-dilated state without transition to a dilated state. In typical embodiments, a TRPV1 ligand is a cannabinoid such as Cannabidiol (CBD), but is not limited to cannabinoids.

“Pharmaceutically active ingredient” (synonymously, active pharmaceutical ingredient) means any substance or mixture of substances intended to be used in the manufacture of a drug product and that, when used in the production of a drug, becomes an active ingredient in the drug product. Such substances are intended to furnish pharmacological activity or other direct effect in the diagnosis, cure, mitigation, treatment or prevention of disease or to affect the structure and function of the body. Such substances or mixture of substances are preferably generated in compliance with the Current Good Manufacturing Practice (CGMP) regulations pursuant to Section 501(a)(2)(B) of the Federal Food, Drug, and Cosmetic Act.

A pharmaceutically active ingredient is “substantially free of THC” if the ingredient contains less than 0.3% (w/w) of delta-9 tetrahydrocannabinol. A pharmaceutical composition is “substantially free of THC” if the pharmaceutical composition contains less than 0.3% (w/v) of delta-9 tetrahydrocannabinol.

A “Cannabis sativa extract” is a composition obtained from Cannabis sativa plant materials by fluid and/or gas extraction, for example by supercritical fluid extraction (SFE) with CO₂. The Cannabis sativa extract typically contains terpenes, cannabinoids, and secondary metabolites. For example, the Cannabis sativa extract can include one or more of terpinene, caryophyllene, geraniol, guaiol, isopulegoll, ocimene, cymene, eucalyptol, and terpinolene.

“Pain disorders” include various diseases causing pain as one of their symptoms—including, but not limited to, those associated with strains, sprains, arthritis or other joint pain, bruising, backaches, fibromyalgia, endometriosis, pain after surgery, diabetic neuropathy, trigeminal neuralgia, postherpetic neuralgia, cluster headaches, psoriasis, irritable bowel syndrome, chronic interstitial cystitis, vulvodynia, trauma, musculoskeletal disorders, shingles, sickle cell disease, heart disease, cancer, stroke, or mouth sores due to chemotherapy or radiation.

The terms “treatment,” “treating,” and the like are used herein to generally mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any conditions can be readily assessed according to standard methods and techniques known in the art. The population of subjects treated by the method includes subjects suffering from the undesirable condition or disease, as well as subjects at risk for development of the condition or disease.

By the term “therapeutically effective dose” or “therapeutically effective amount” is meant a dose or amount that produces the desired effect for which it is administered. The exact dose or amount will depend on the purpose of the treatment, and will be ascertainable by one skilled in the art using known techniques (see, e.g., Lloyd (2012) The Art, Science and Technology of Pharmaceutical Compounding, Fourth Edition). A therapeutically effective amount can be a “prophylactically effective amount” as prophylaxis can be considered therapy.

The term “sufficient amount” means an amount sufficient to produce a desired effect.

The term “ameliorating” refers to any therapeutically beneficial result in the treatment of a disease state, e.g., an immune disorder, including prophylaxis, lessening in the severity or progression, remission, or cure thereof.

### Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereo centers intends each stereoisomer, and all combinations of stereoisomers, thereof.

### Overview of Experimental Results

Cannabis has been used for millennia to provide analgesia and treat various types of pain. In the earlier studies described in U.S. application Ser. No. 15/986,316 and PCT/US2018/033956, which are incorporated by reference in their entireties and further described herein, we demonstrated that Cannabis exerts its anti-nociceptive effects at least in part through the TRPV1 receptor. We further demonstrated that myrcene contributes significantly to the observed TRPV1 agonism, and that like capsaicin, causes TRPV1 desensitization after prolonged exposure.

In this disclosure, we sought to identify additional terpene/cannabinoid compounds in a medicinal Cannabis that contribute to the TRPV1 desensitization and anti-nociceptive effects.

As described more fully in the Example section below, we prepared a complex mixture of cannabinoids and terpenes, the Strain A Mixture, based upon the actual chemo-profile of a Cannabis sativa cultivar currently used medicinally in Nevada, USA. Strain chemo-profile data was expressed as % mass and mg/g abundance, and these amounts were converted to amounts to be included in the mixture. The actual chemo-profile was modified in the Strain A Mixture by deliberate omission of THC and THCA, to eliminate psychoactive components, and omission of certain labile or insoluble components. We also prepared complex mixtures containing subsets of the compounds in the Strain A Mixture: CBMIX, Cannabinoid Mixture and Terpene Mixture. See Table 1.

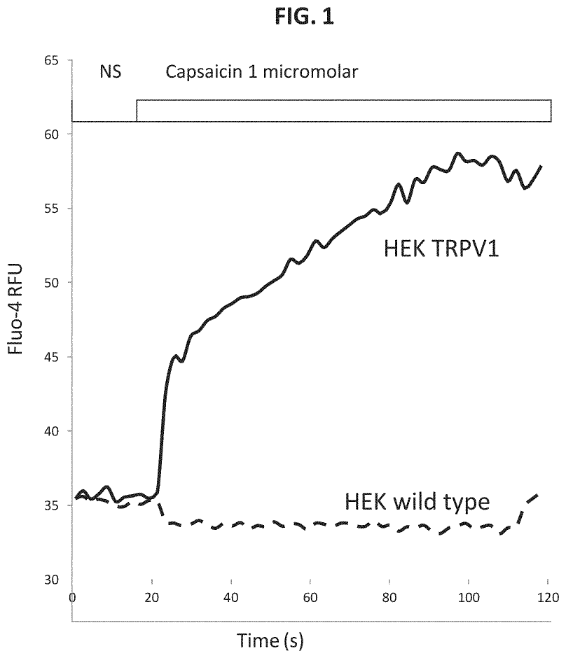

To create an in vitro assay for TRPV-1 agonist activity, we transfected HEK cells with an expression vector that confers tetracycline-inducible expression of TRPV1 on the cells, and used a standard fluorescent reporter of intracellular calcium levels (fluo-4 acetoxymethyl ester) (“fluo-4”). FIG. 1 illustrates that the inducible expression of TRPV1 confers capsaicin-sensitive calcium flux responses upon HEK cells, establishing that the experimental system clearly reports TRPV1-specific calcium fluxes.

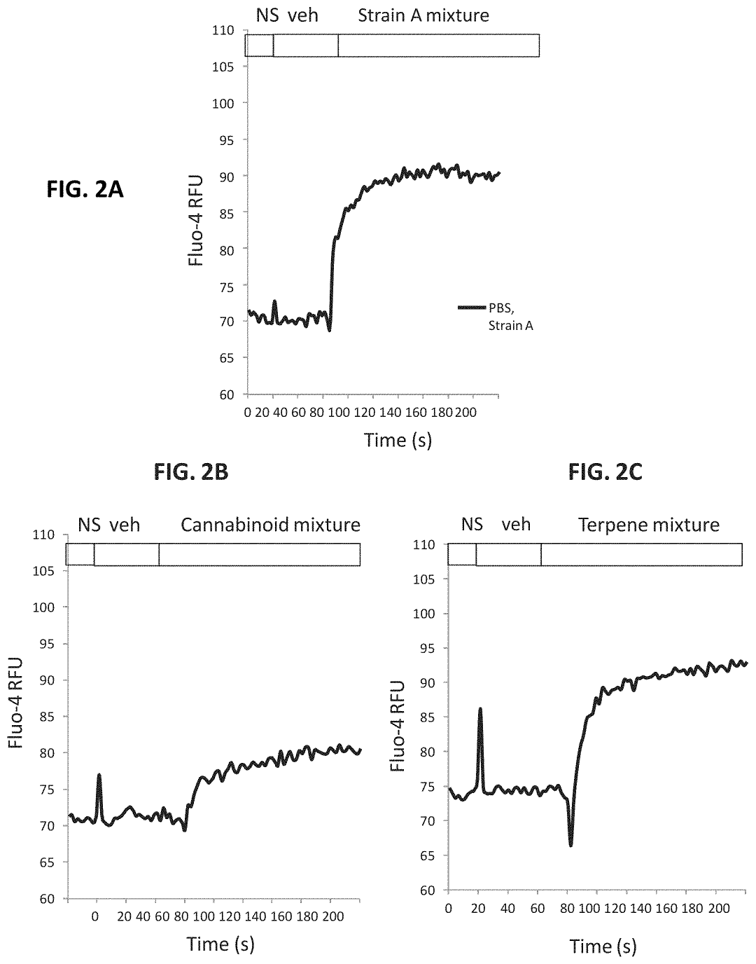

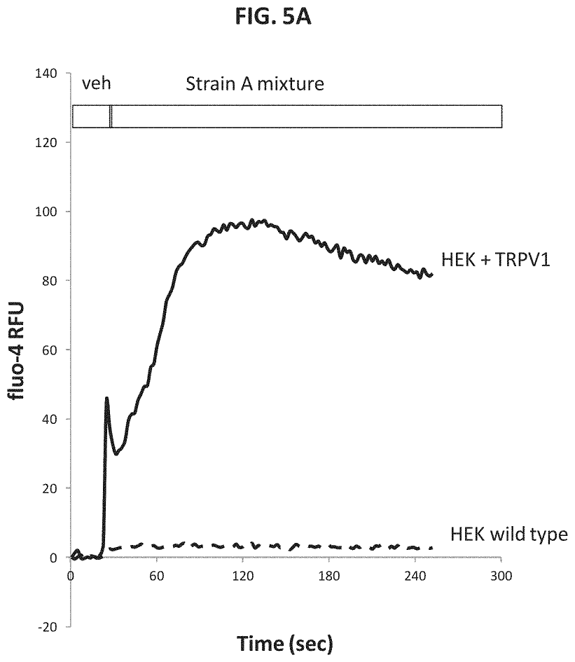

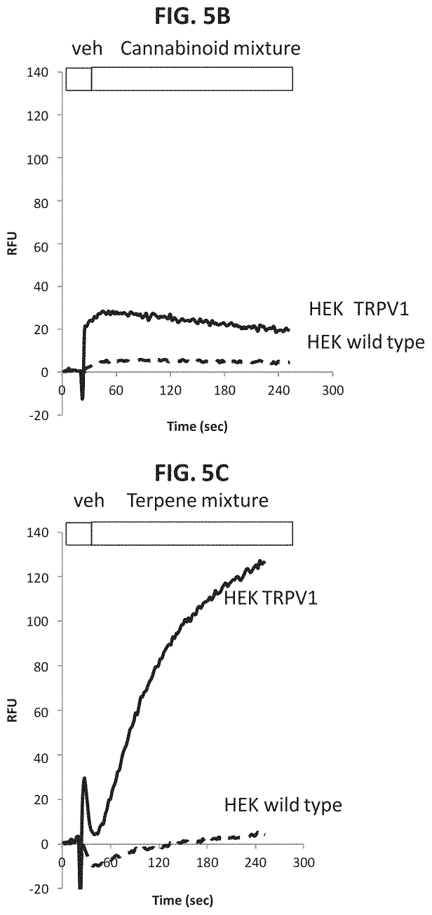

We tested the Strain A mixture in the same assay, and found that the Cannabis-derived mixture of cannabinoids and terpenes (Strain A mixture) causes significant calcium flux into the TRPV1-transfected HEK cells (FIG. 2A). We confirmed that the calcium fluxes observed with the complete Strain A mixture depends on the presence of the TRPV1 receptor by comparing signals obtained in parallel with untransfected wild type HEK cells (FIG. 5A).

Using sub-mixtures, we determined that the terpenes in the Strain A mixture contribute significantly to the observed effect (FIG. 2C). More modest influx was caused by the cannabinoids present in the Strain A mixture (FIG. 2B). The signal observed using the Terpene Mixture and the Cannabinoid Mixture were dependent on the presence of the TRPV1 receptor (FIGS. 5B and 5C).

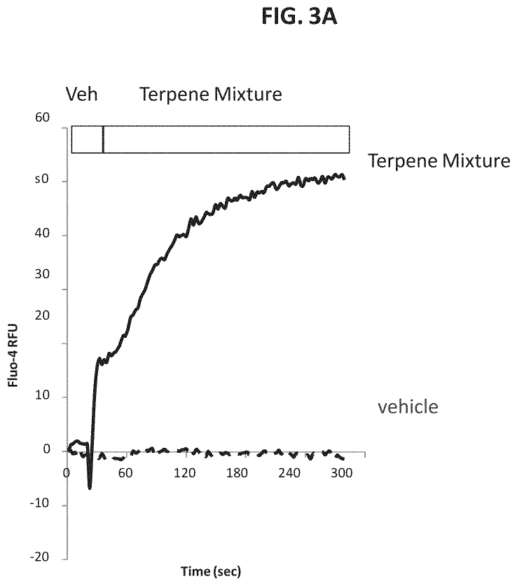

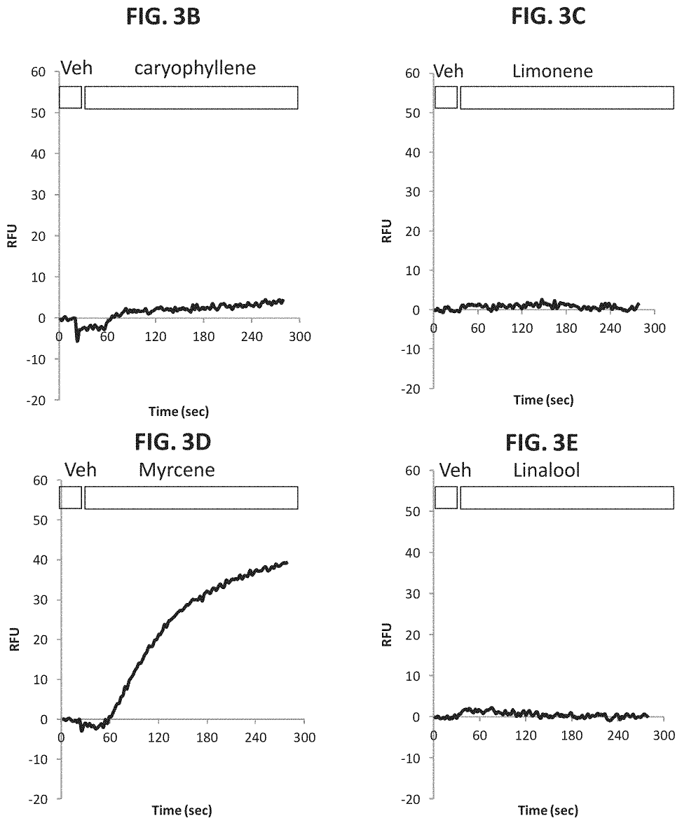

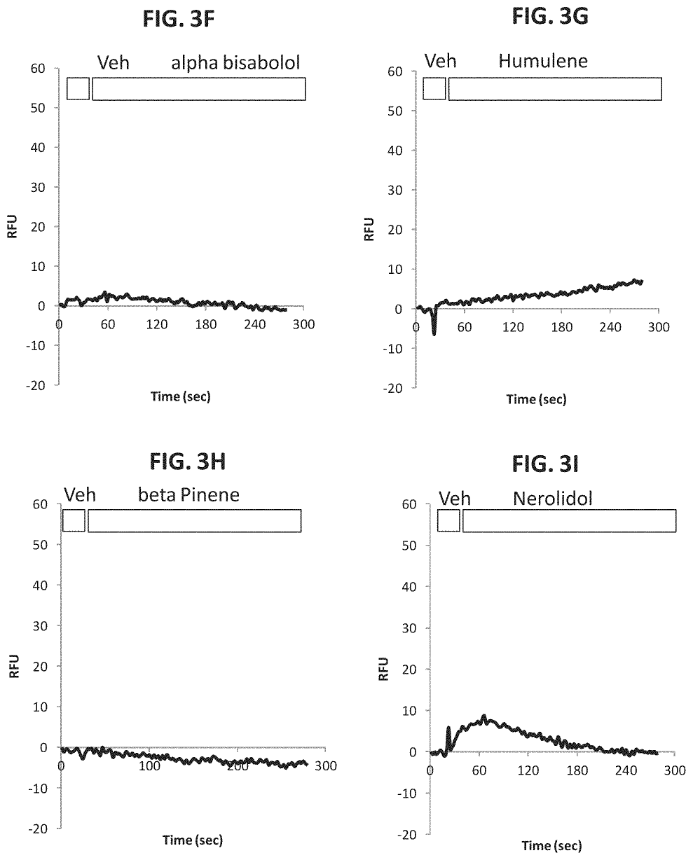

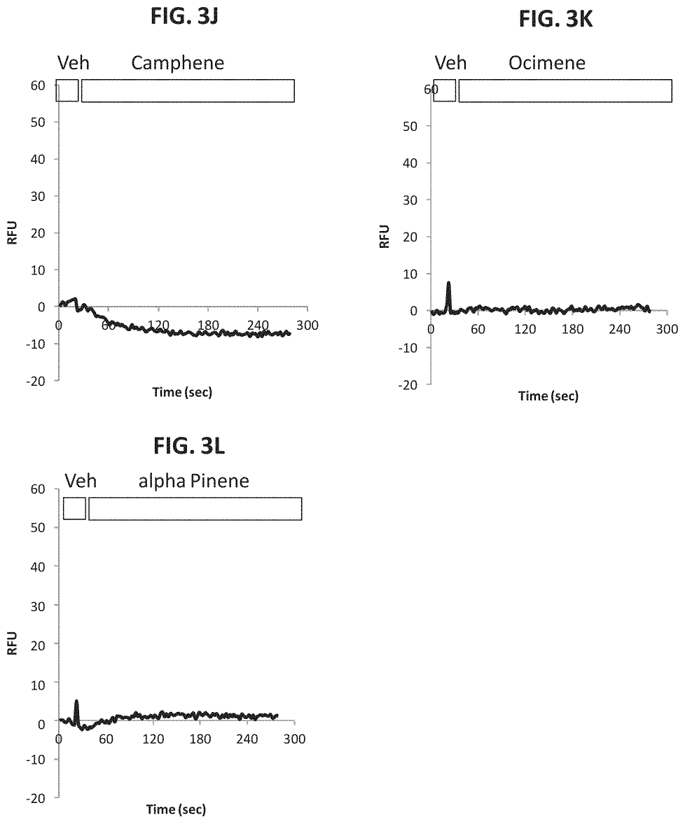

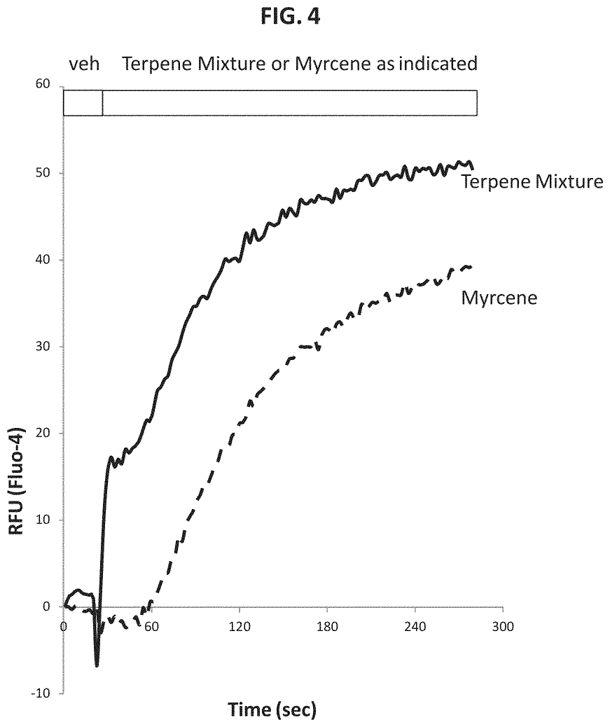

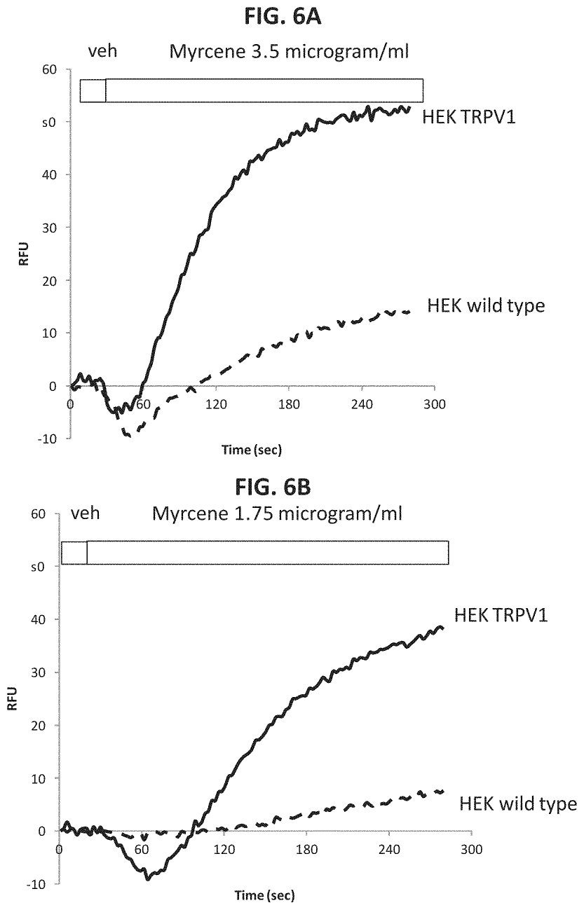

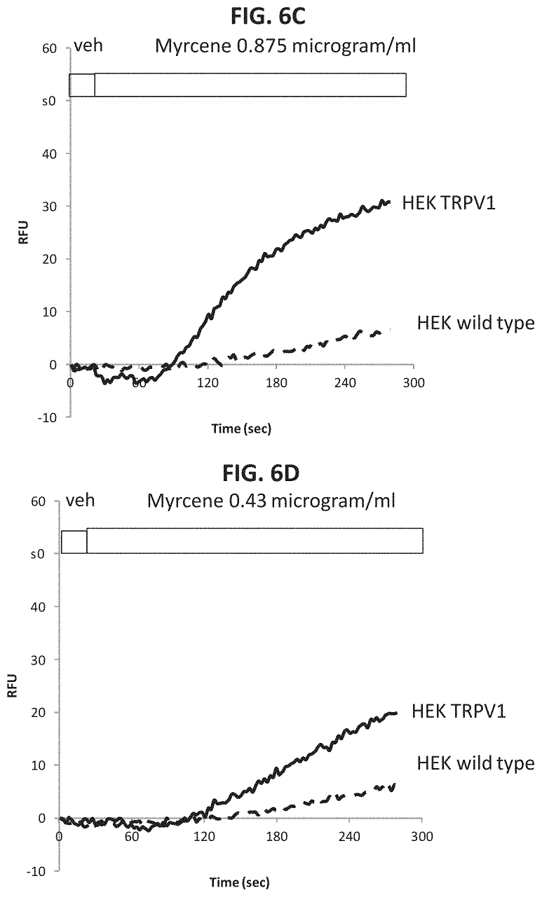

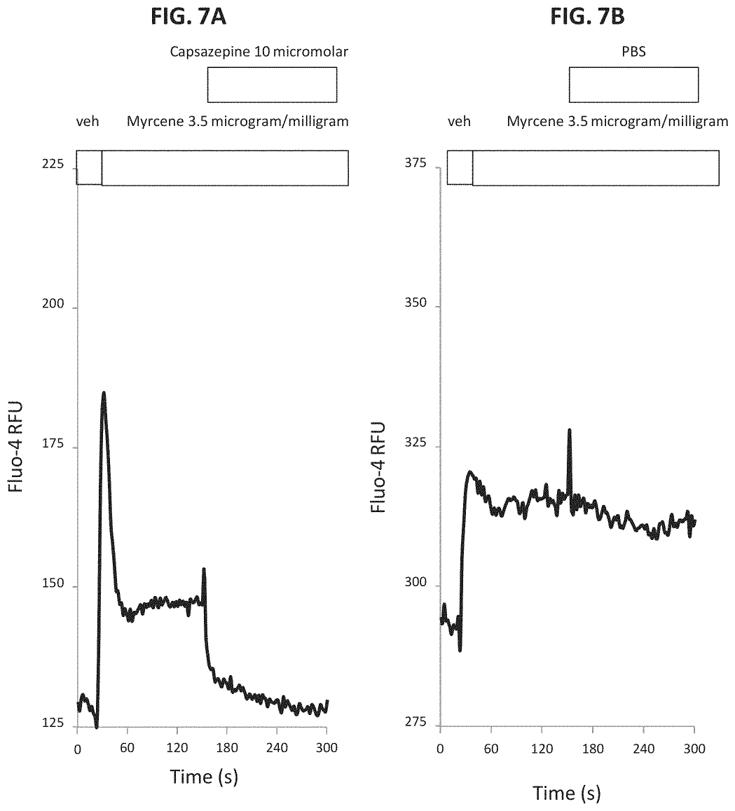

We then tested individual terpenes present in the Strain A mixture and found that individual terpenes differentially contribute to calcium fluxes via TRPV1 (FIGS. 3A-3L). Myrcene contributes significant agonist activity (FIG. 3C), but does not constitute 100% of the signal (FIG. 4). Nerolidol was observed to have more modest agonist activity when tested alone (FIG. 3I). The myrcene-induced influx of calcium was dose-dependent, and dependent wholly or in part on expression of TRPV1 receptors (FIG. 6). We further confirmed the dependence on TRPV1 using the TRPV1 inhibitor, capsazepine; FIG. 7 illustrates that the myrcene-induced calcium influx responses are inhibited by capsazepine.

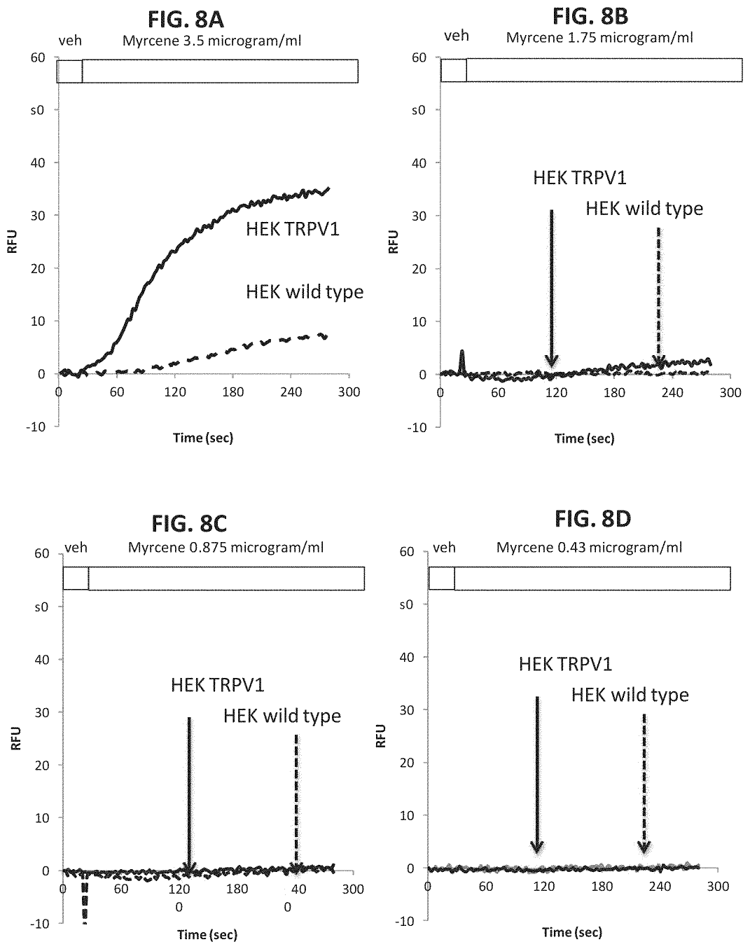

We demonstrated that in the absence of extracellular calcium (achieved by construction of a nominally calcium-free extracellular milieu supplemented with 1 mM EGTA), at high concentrations myrcene can induce TRPV1-dependent calcium release from internal stores (FIG. 8).

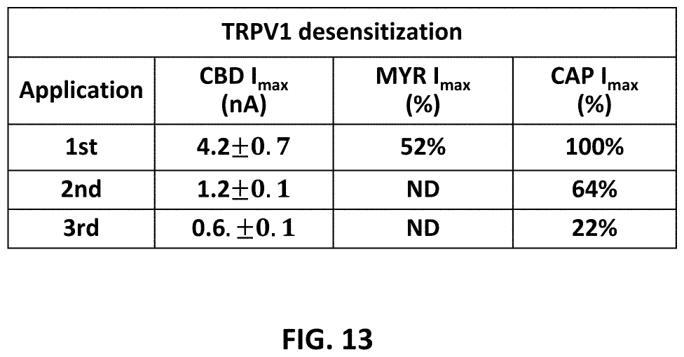

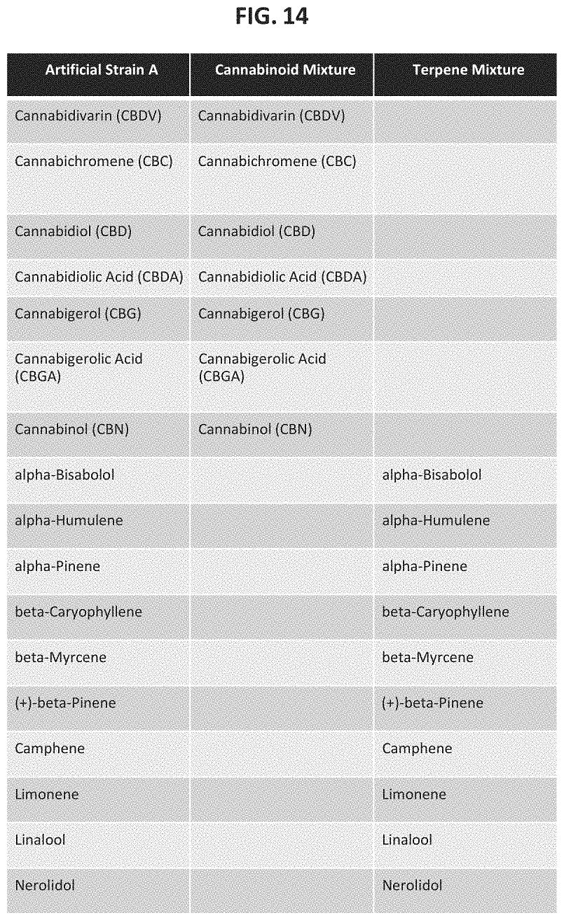

Data summarized in FIG. 13 further shows that compounds that target sites 4 or 4A can desensitize TRPV1. FIG. 13 shows that CBD and MYR cause desensitization of the channel measured by Area Under the Curve analysis, calcium assays or electrophysiology methods.

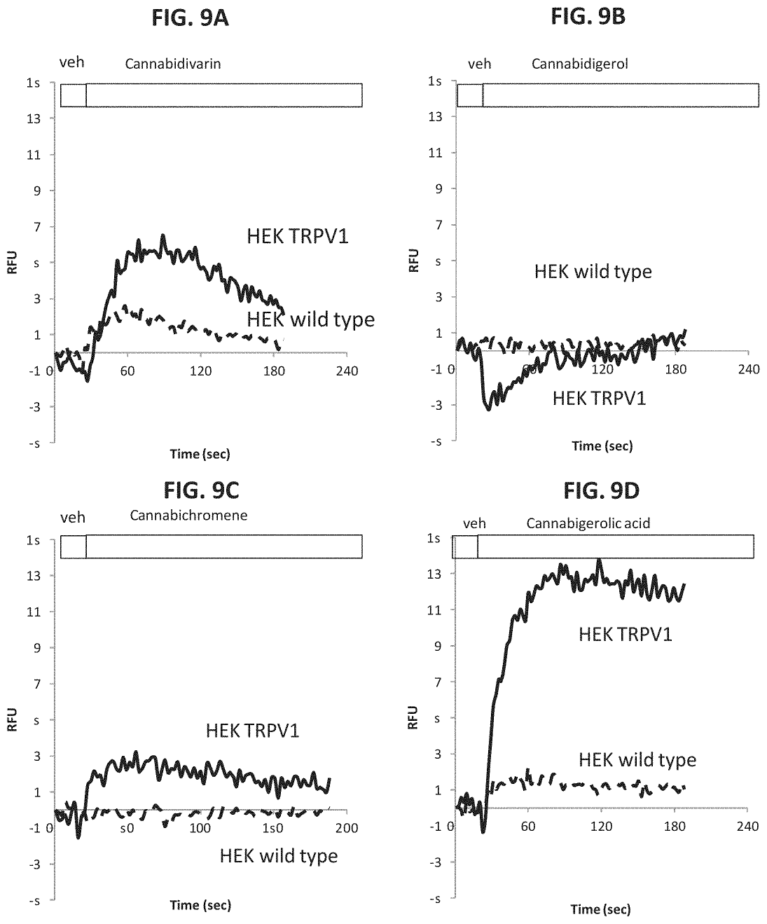

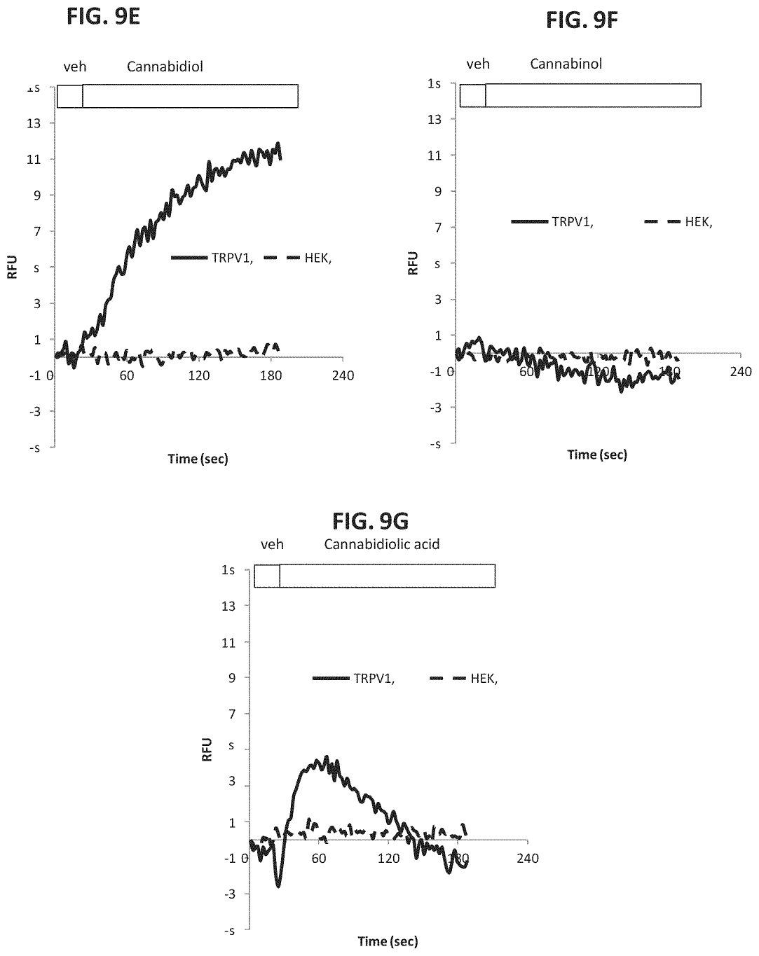

We performed similar experiments to determine the contribution of individual cannabinoids in the Strain A mixture, and found that that cannabinoids differentially contribute to calcium fluxes via TRPV1 (FIGS. 9A-9G). Of the cannabinoids, cannabigerolic acid (CBGA), cannabidiol (CBD), cannabidivarin (CBDV), cannabichromene (CBC), and cannabidiolic acid (CBDA) were most potent when tested individually in the assay,

In order to assess the broader therapeutic potential of myrcene and nerolidol, the two terpenes in our original Cannabis Strain A Mixture with significant TRPV1 agonist effects, we used a proprietary in silico prediction approach, termed the GB Sciences' Network Pharmacology Platform (“NPP”).

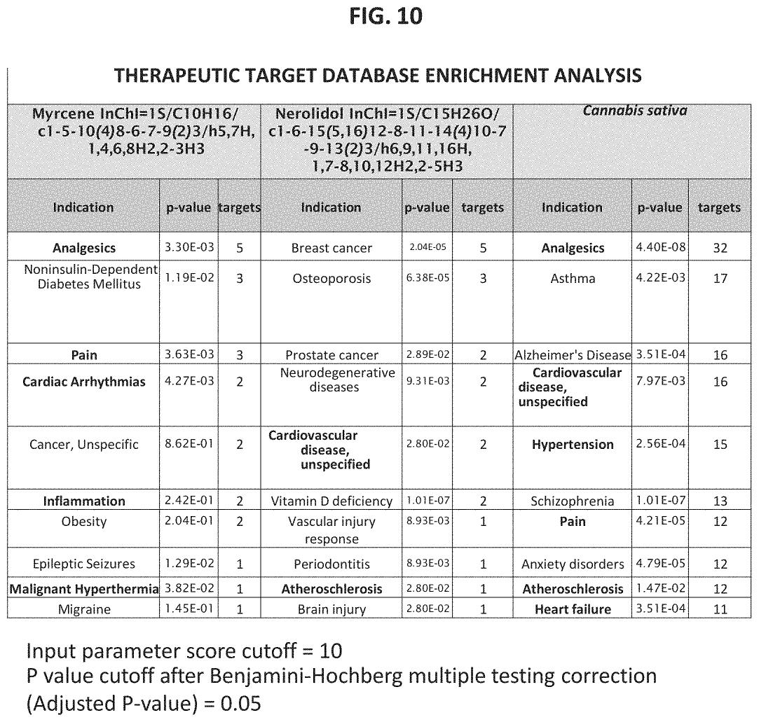

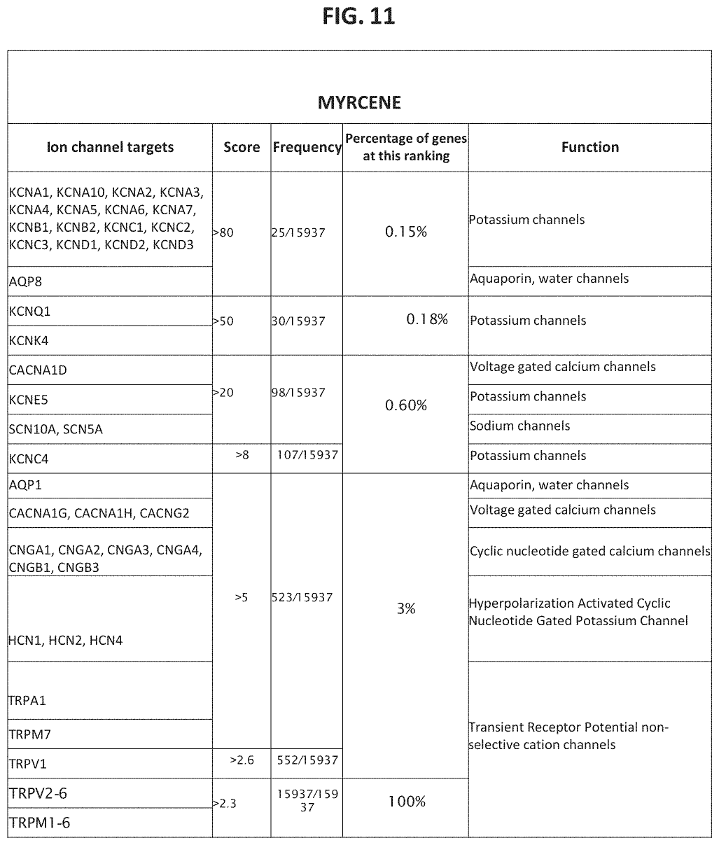

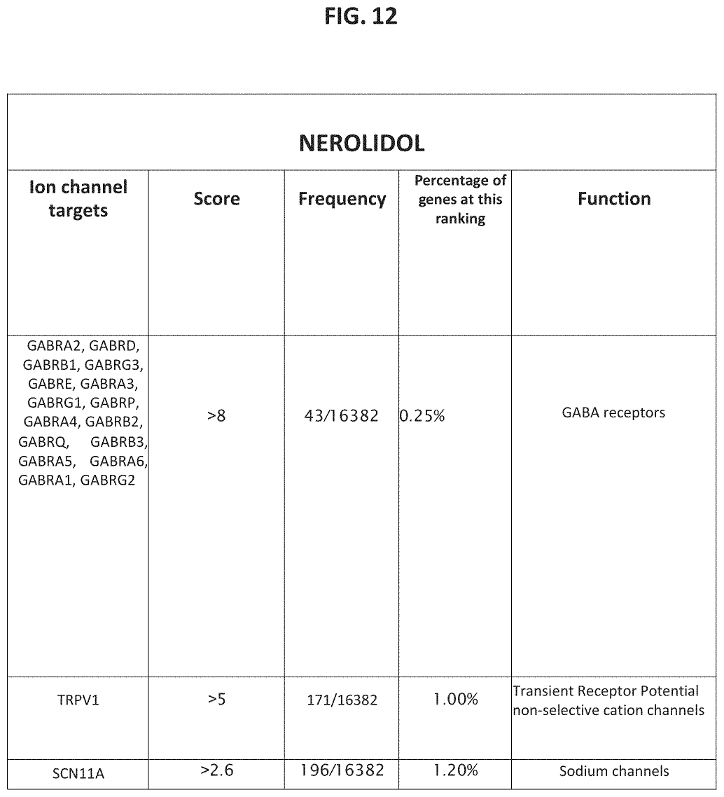

FIG. 10 illustrates that Therapeutic Target Database enrichment analysis tends to prioritize myrcene over nerolidol for development in pain and cardiovascular areas. In addition, myrcene contributes significantly to the predicted disease target set for native Cannabis. FIG. 11 illustrates that diverse ion channel targets are predicted for direct or indirect modulation by myrcene, whereas FIG. 12 illustrates that a more limited set of ion channel targets or CNS-active targets are predicted for direct or indirect modulation by nerolidol

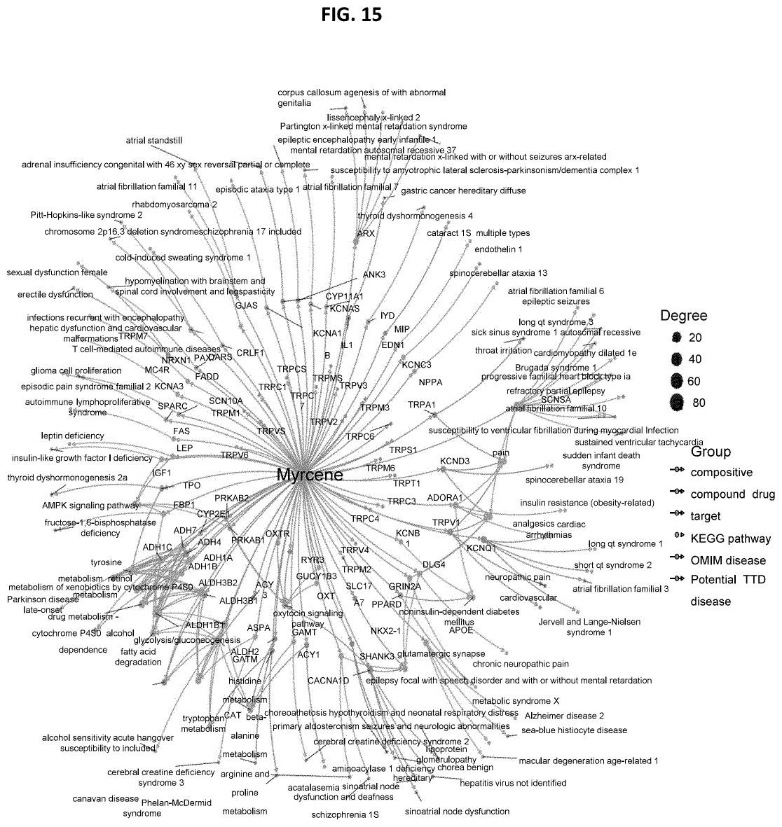

FIG. 15 shows a target analysis and disease-prediction network for myrcene using GB Sciences' NPP. The presence of multiple TRP channels in the network indicates that efficacy of myrcene will likely extend beyond TRPV1 to other nociceptive neurons in which the primary pain conduction channel is a distinct TRP receptor.

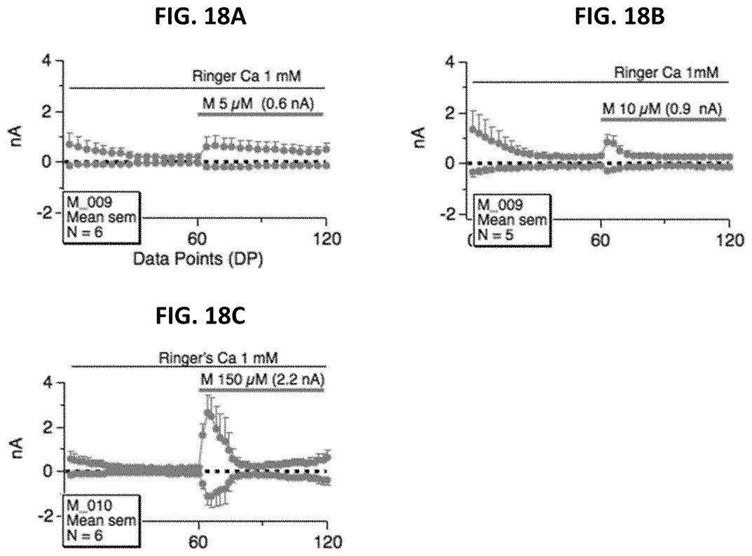

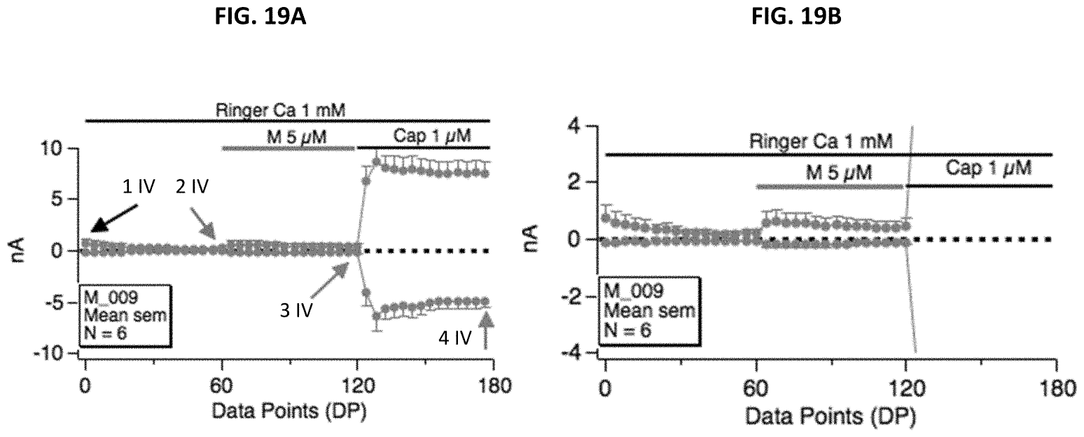

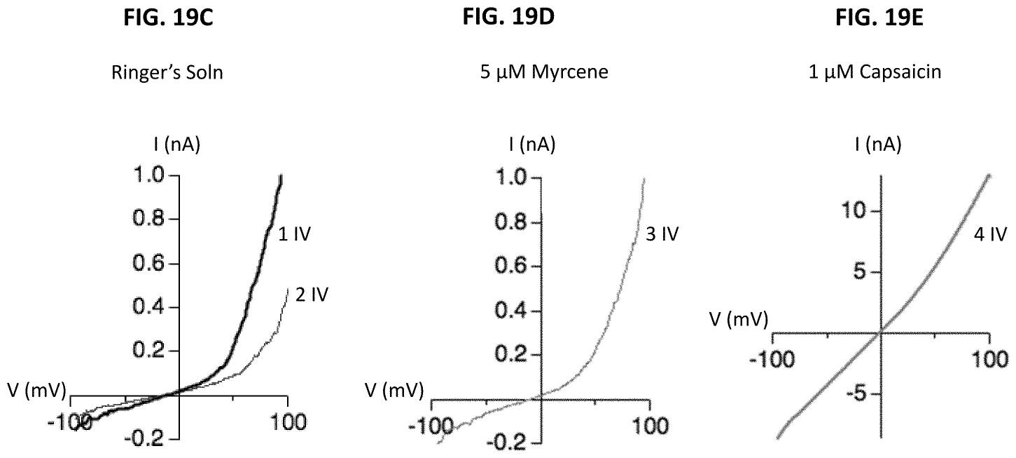

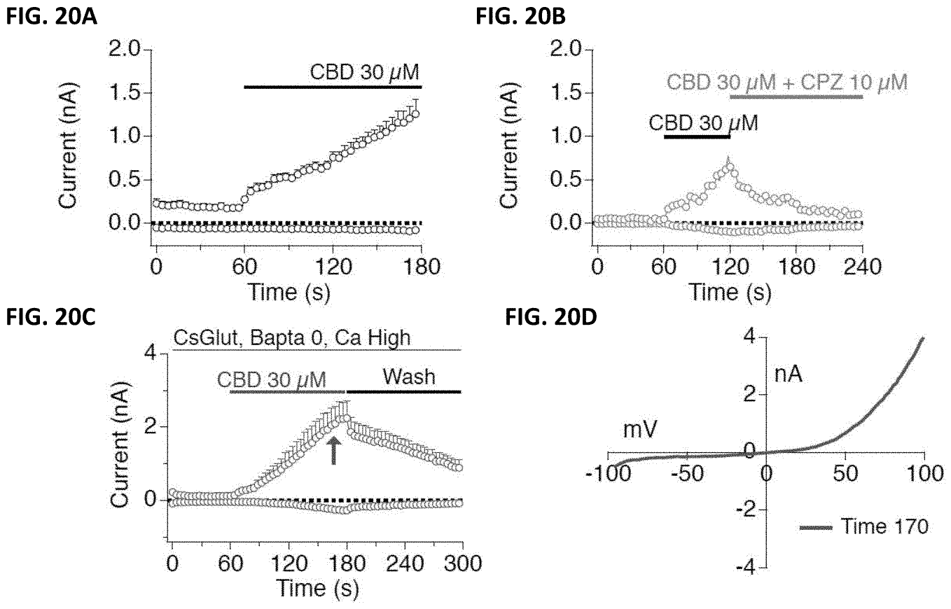

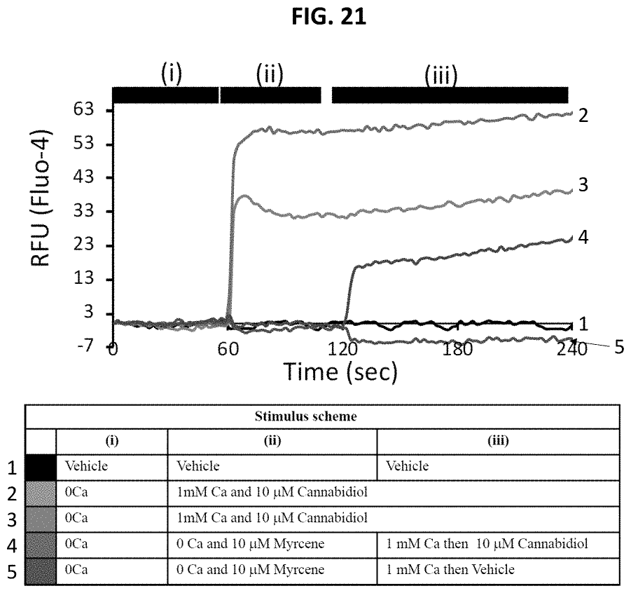

To further study modulation of TRPV1 activity by myrcene, we performed patch-clamp experiments. First, we tested a dose-dependent response in individual cells expressing TRPV1 and found that increasing doses of myrcene result in an inwardly rectifying non-selective cation current which can be inactivated in a manner dependent both on activation current amplitude (FIGS. 18A-18C) and calcium influx (data not shown). Assessment of IV relationship in FIG. 19A before and after addition of Myrcene and Capsaicin (at 1IV, 2IV, 3IV and 4IV), as provided in FIGS. 19C-E, showed that Myrcene activates TRPV1 primarily in State 1 (undilated), whereas Capsaicin activated TRPV1 to a dilated state. TRPV1 was also activated by Cannabidiol (CBD) (FIG. 20A), initiating the current sensitive to both capsazepine and washout (FIGS. 20B and 20C). Application of Myrcene could modulate subsequent activation of TRPV1 by CBD (FIG. 21), suggesting that Myrcene has the potential to act as an allosteric modulator of other TRPV1 ligands.

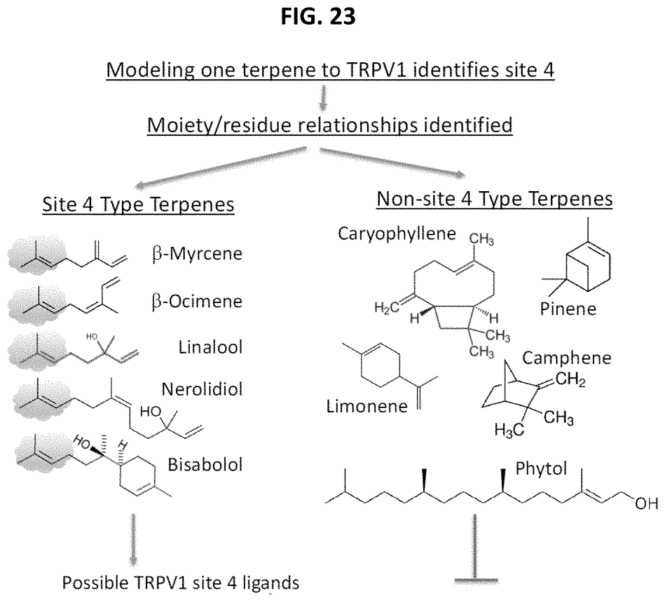

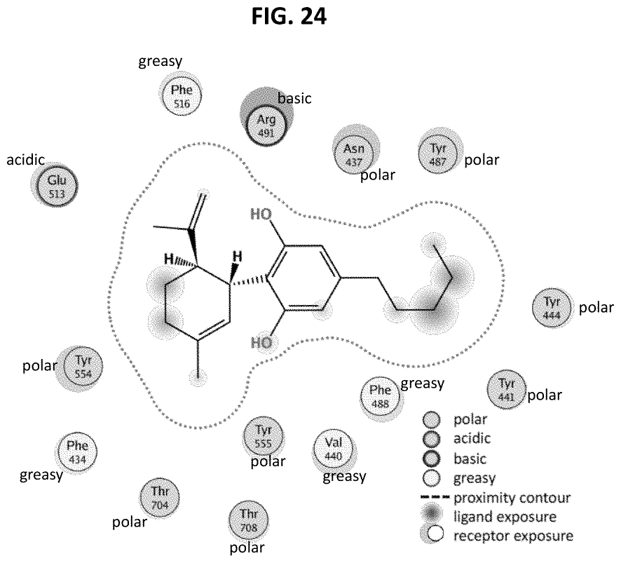

An unbiased computational modeling analysis allowed us to identify a binding pocket (site 4) for Myrcene in TRPV1 as illustrated in FIG. 22A-22B. The binding pocket comprises a set of amino acid residues including: Arg 491, Asn 437, Phe 434, Tyr 555, Ser 512, Tyr 554, Glu 513, Phe 516, and Phe 488 of TRPV1 (FIGS. 23A and 23B). The identified binding pocket was used a tool to identify additional compounds that exert allosteric effects on TRPV1, similar to Myrcene. Based on chemical structures of various terpenes (FIG. 23) and their predicted interactions with site 4, it was predicted that terpenes comprising a dimethylallyl group (e.g., β-ocimene, linalool, nerolidol, and bisabolol) would bind to the site 4. It was also predicted that terpenes without a dimethylallyl group (e.g., caryophyllene, pinene, limonene, camphene, and phytol.) would not bind to the site 4.

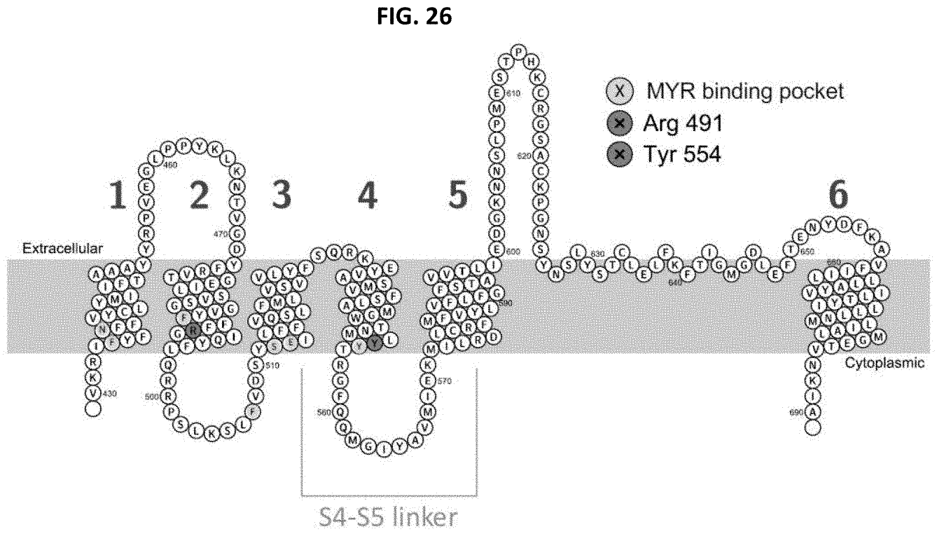

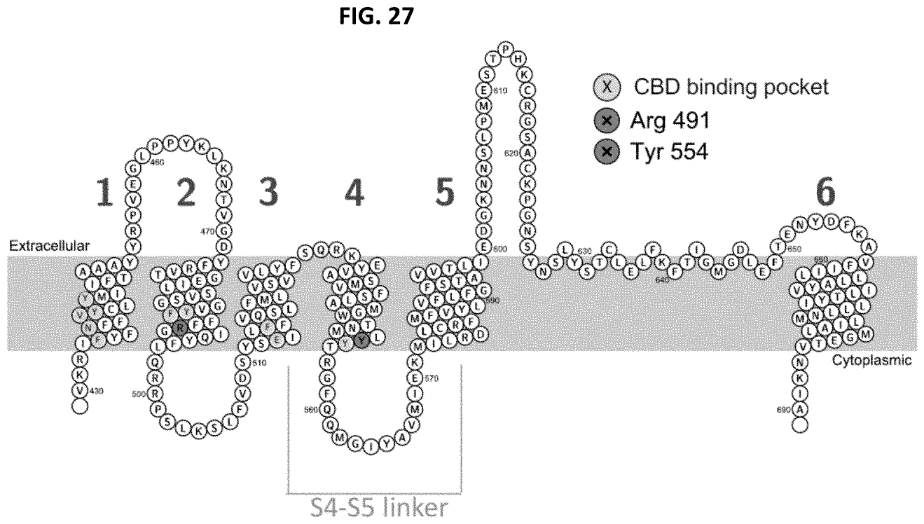

The unbiased computational modeling analysis further allowed us to identify a binding pocket (site 4A) for Cannabidiol (CBD) in TRPV1 as illustrated in FIG. 25A-25B. The binding pocket comprises a set of amino acid residues including: Arg 491, Asn 437, Tyr 487, Tyr 444, Tyr 441, Phe 488, Val 440, Tyr 555, Thr 708, Thr 704, Phe 434, Tyr 554, Glu 513, and Phe 516. The binding pocket (site 4A) partially overlapped with but was different from that of Myrcene (site 4). Y554 and R491 appear to provide key interactions with CBD, similar to Myrcene, but the remaining residues implicated in CBD binding show both similarities and differences to the Myrcene site. The residues implicated in the bindings of Myrcene and CBD across a two-dimensional representation of the channel's protein sequence is shown in FIGS. 26 and 27.

### Methods of Designing Complex Mixture for Treatment

In one aspect, the present disclosure provides a new method of designing a complex mixture for treating pain through targeting a TRP channel selected from TRPV1, TRPV2, TRPM8 and TRPA1. The method involves the steps of analyzing compounds in Cannabis or other plants using in vitro or in silico technique and predicting whether each of the compounds binds to site 4 or site 4A of TRPV1, and assessing their relative binding energies, thereby differentiating between likely analgesic and non-analgesic compounds; selecting a subset of the compounds that contain a functional dimethyl moiety and excluding a different subset of the compounds that do not contain the functional dimethyl moiety, thereby obtaining selected compounds; and designing the complex mixture comprising the selected compounds. The method can further comprise the step of identifying one or more compounds that do not initiate state transition or pore dilation in TRPV1. Compounds that do not initiate state transition can be identified by methods known in the art, for example, whole cell patch clamping, other electrophysiology techniques, calcium imaging, or other methods that allow identification of signals specific to the state transition of TRPV1.

In some embodiments, the methods can be applied to identify an allosteric modulator of TRPV1 among natural or synthetic compounds available in the art. In preferred embodiments, the methods are applied to identify an allosteric modulator of TRPV1 among terpenes and cannabinoids found in Cannabis. In other embodiments, the methods can be used to design and synthesize a new compound that can modulate TRPV1 activity and provide the desired therapeutic effects, e.g., analgesic effects.

The step of analyzing various compounds using in vitro or in silico techniques and predicting whether each of the compounds binds to site 4 or site 4A of TRPV1, and with what relative binding energy, can be used to screen a large number of compounds to select a smaller number of compounds that can be further tested for their TRPV1 modulatory and analgesic effects. In some embodiments, the step of analyzing various compounds using in vitro or in silico technique and predicting whether each of the compounds binds to site 4 or site 4A of TRPV1 can be used to study physiological effects of compounds identified or suspected to have modulatory effects on TRPV1.

The complex mixture can be designed to include one or more compound identified to bind to site 4 or site 4A of TRPV1 using in vitro or in silico technique. In some embodiments, the complex mixture includes only one compound identified to bind to site 4 or site 4A of TRPV1 using in vitro or in silico technique. In some embodiments, the complex mixture includes a first compound identified to bind to site 4 and a second compound identified to bind to site 4A of TRPV1 using in vitro or in silico technique. The compound(s) to be included in the complex mixture can be selected based on their binding affinities to site 4 or site 4A of TRPV1. In some embodiments, compound(s) to be included in the complex mixture can be selected based on specific amino acid residues of site 4 that are interacting or predicted to interact with the compounds.

In some embodiments, compounds are selected only when they contain a functional dimethyl moiety. In some embodiments, compounds that do not contain a functional dimethyl moiety are excluded during the screening process.

### Methods of Treatment

### Methods of Treating Pain in a Mammalian Subject

We have identified terpene and cannabinoid compounds having acute agonistic effects and long-term desensitization effects on TRPV1. The compounds bind to site 4 or site 4A of TRPV1, binding sites that partially overlap but are distinct from binding sites to capsaicin. This suggests that the terpene and cannabinoid compounds have different physiological effects on TRPV1 from capsaicin. In particular, they may sustain the activated TRPV1 channel in a specific non-dilated state without transition to dilated state, and thus different pharmaceutical potential to capsaicin, for example in causing analgesia absent cytotoxic effects in sensory neurons.

Accordingly, the present disclosure provides the methods of effecting TRPV1 desensitization in cells of a mammalian subject, the method comprising administering to the subject a pharmaceutical composition, in an amount, by a route of administration, and for a time sufficient to cause TRPV1 inactivation or desensitization in sensory neurons within the subject, wherein the pharmaceutical composition comprises an active compound capable of activating TRPV1 by binding to site 4 or 4A of TRPV1, and a pharmaceutically acceptable carrier or diluent; and wherein the active compound is (i) a naturally occurring compound, optionally a Cannabis-derived compound, or (ii) a synthetic compound.

In various embodiments, the pharmaceutical composition is administered topically.

In various embodiments, the pharmaceutical composition is administered systemically. In some embodiments, the pharmaceutical composition is administered orally, by buccal administration, or sublingually.

In some embodiments, the pharmaceutical composition is administered parenterally. In certain embodiments, the pharmaceutical composition is administered intravenously. In some embodiments, the pharmaceutical composition is administered subcutaneously. In some embodiments, the pharmaceutical composition is administered by inhalation.

These methods are particularly aimed at therapeutic and prophylactic treatments of mammals, and more particularly, humans.

The actual amount administered, and rate and schedule of administration, will depend on the nature and severity of disease being treated. Prescription of treatment, e.g. decisions on dosage etc., is within the responsibility of general practitioners and other medical professionals, and typically takes account of the disorder to be treated, the condition of the individual patient, the route of administration, the site to be treated, and other factors known to practitioners. Examples of the techniques and protocols mentioned above can be found in Remington's Pharmaceutical Sciences, 16th edition, Osol, A. (ed), 1980.

In vivo and/or in vitro assays may optionally be employed to help identify optimal dosage ranges for use and routes and times for administration. The precise dose to be employed in the formulation will also depend on the route of administration, and the seriousness of the condition, and should be decided according to the judgment of the practitioner and each subject's circumstances. Effective doses and methods of administration may be extrapolated from dose-response curves derived from in vitro or animal model test systems.

In some embodiments, an allosteric modulator of TRPV1 is administered in an amount less than 1 g, less than 500 mg, less than 100 mg, less than 10 mg per dose.

In the methods of treatment described herein, the pharmaceutical composition can be administered alone or in combination with other treatments administered either simultaneously or sequentially with the composition.

### Methods of Treating Pain

In some embodiments, the cells to be subjected to TRPV1 desensitization are sensory neurons, and the method comprises administering to the subject the pharmaceutical compositions described herein in an amount, by a route of administration, and for a time sufficient to cause TRPV1 desensitization in the sensory neurons within the subject.

In some embodiments, the sensory neurons are nociceptive neurons. In some embodiments, the sensory neurons are peripheral nociceptive neurons. In some embodiments, the sensory neurons are visceral nociceptive neurons.

Thus, the present disclosure further provides the method of treating pain in a mammalian subject, comprising: administering to the subject a pharmaceutical composition, in an amount, by a route of administration, and for a time sufficient to cause TRPV1 inactivation or desensitization in sensory neurons within the subject, wherein the pharmaceutical composition comprises an active compound capable of activating TRPV1 by binding to site 4 or 4A of TRPV1, and a pharmaceutically acceptable carrier or diluent; and wherein the active compound is (i) a naturally occurring compound, optionally a Cannabis-derived compound, or (ii) a synthetic compound.

In some embodiments, the method of treating pain comprises administering to the subject a pharmaceutical composition, in an amount, by a route of administration, and for a time sufficient to cause TRPV1 inactivation or desensitization in sensory neurons within the subject, wherein the pharmaceutical composition comprises (i) an allosteric modulator capable of activating TRPV1 by binding to site 4 of TRPV1, (ii) a TRPV1 ligand capable of activating TRPV1 by binding to a ligand-binding site at least partially overlapping with the site 4 of TRPV1, and (iii) a pharmaceutically acceptable carrier or diluent, wherein each of the allosteric modulator and the TRPV1 ligand is naturally occurring, optionally Cannabis-derived, or synthesized; and wherein the allosteric modulator and the TRPV1 ligand are different compounds.

Our in silico analyses using the GB Sciences Network Pharmacology Platform, described below in Example 8, indicates that therapeutic efficacy of the pharmaceutical composition or its active compound will likely extend beyond TRPV1 to other nociceptive neurons in which the primary pain-conducting channel is a distinct TRP and other diseases in which these channels play a role, identified using the compound-to-gene-to disease approach in the NPP. Accordingly, in a related aspect, methods are provided for treating pain in a mammalian subject by targeting TRPV1 or other TRP channels. The method comprises administering to the subject the pharmaceutical compositions described herein in an amount, by a route of administration, and for a time sufficient to reduce pain.

In certain embodiments, the pain is neuropathic pain. In some embodiments, the neuropathic pain is diabetic peripheral neuropathic pain. In some embodiments, the pain is post-herpetic neuralgia. In some embodiments, the pain is trigeminal neuralgia.

In some embodiments, the subject has pain related to or caused by strains, sprains, arthritis or other joint pain, bruising, backaches, fibromyalgia, endometriosis, surgery, migraine, cluster headaches, psoriasis, irritable bowel syndrome, chronic interstitial cystitis, vulvodynia, trauma, musculoskeletal disorders, shingles, sickle cell disease, heart disease, cancer, stroke, or mouth sores or ulceration due to chemotherapy or radiation.

In some embodiments, the pharmaceutical composition is administered at least once a day for at least 3 days. In some embodiments, the pharmaceutical composition is administered at least once a day for at least 5 days. In some embodiments, the pharmaceutical composition is administered at least once a day for at least 7 days. In some embodiments, the pharmaceutical composition is administered at least once a day for more than 7 days.

In various embodiments, the pharmaceutical composition is administered at a dose, by a route of administration, and on a schedule sufficient to maintain effective levels of the active compound (i.e., the allosteric modulator or the TRPV1 ligand) at the nociceptors for at least 3 days, at least 5 days, or at least 7 days.

In some embodiments, the pharmaceutical composition is administered topically, systemically, intravenously, subcutaneously, or by inhalation.

### Methods of Treating Cardiac Hypertrophy

In another aspect, methods of treating cardiac hypertrophy in a mammalian subject are provided. The methods comprise administering to the subject an anti-hypertrophic effective amount of the pharmaceutical compositions described herein.

In typical embodiments, the pharmaceutical composition is administered systemically.

In some embodiments, the pharmaceutical composition is administered intravenously. In some embodiments, the pharmaceutical composition is administered subcutaneously. In some embodiments, the pharmaceutical composition is administered by inhalation. In some embodiments, the pharmaceutical composition is administered orally.

### Methods of Prophylactic Treatment for Cardiac Hypertrophy

In another aspect, methods of prophylactic treatment for cardiac hypertrophy in a mammalian subject are provided. The methods comprise administering to a subject at risk of cardiac hypertrophy an anti-hypertrophic effective amount of the pharmaceutical compositions described herein.

### Methods of Treating Overactive Bladder

In another aspect, methods of treating overactive bladder in a mammalian subject, are provided. The methods comprise administering to the subject a therapeutically effective amount of the pharmaceutical compositions described herein.

In typical embodiments, the pharmaceutical composition is administered systemically.

### Methods of Treating Refractory Chronic Cough

In another aspect, methods of treating refractory chronic cough are provided, the methods comprising administering to the subject a therapeutically effective amount of the pharmaceutical composition described herein.

In some embodiments, the pharmaceutical composition is administered systemically.

In some embodiments, the pharmaceutical composition is administered by inhalation.

Methods of Treating Disorders with TRPV1 Etiology

In another aspect, diseases or disorders that are treated with the pharmaceutical compositions described herein include diseases related to abnormal function of TRPV1. The diseases can be related to abnormal activation, suppression, or dysregulation of TRPV1. In some embodiments, the diseases are related to abnormal expression or mutation of the gene encoding TRPV1.

In some embodiments, diseases treated with the pharmaceutical compositions described herein are diseases related to abnormal synthesis of an endogenous TRPV1 agonist.

### Pharmaceutical Compositions

In one aspect, pharmaceutical compositions are provided. The composition comprises an active compound (i.e., an allosteric modulator or TRPV1 ligand) capable of activating TRPV1 by binding to site 4 or site 4A of TRPV1 and a pharmaceutically acceptable carrier or diluent, wherein the composition is substantially free from THC; and wherein the allosteric modulator is a naturally occurring compound, optionally Cannabis-derived compound, or synthesized compound.

In some embodiments, the composition comprises an allosteric modulator capable of activating TRPV1 by binding to site 4 of TRPV1 and a pharmaceutically acceptable carrier or diluent, wherein the composition is substantially free from THC; and wherein the allosteric modulator is a naturally occurring compound, optionally Cannabis-derived compound, or synthesized compound.

In some embodiments, the allosteric modulator binds to a binding pocket of a set of amino acid residues, wherein the amino acid residues comprise: Arg 491, Asn 437, Phe 434, Tyr 555, Ser 512, Tyr 554, Glu 513, Phe 516, and Phe 488 of rat TRPV1 or the closely equivalent human TRPV1 residues (see Table 2). In some embodiments, the allosteric modulator binds to a subset of amino acid residues, wherein the amino acid residues comprise: Arg 491, Asn 437, Phe 434, Tyr 555, Ser 512, Tyr 554, Glu 513, Phe 516, and Phe 488 of TRPV1. In some embodiments, the allosteric modulator binds to 2, 3, 4, 5, 6, 7, 8, or 9 amino acid residues selected from the group consisting of: Arg 491, Asn 437, Phe 434, Tyr 555, Ser 512, Tyr 554, Glu 513, Phe 516, and Phe 488 of rat TRPV1 or the closely equivalent human TRPV1 residues (see Table 2).

In some embodiments, the pharmaceutical composition further comprises a TRPV1 ligand capable of activating TRPV1 by binding to a ligand-binding site at least partially overlapping with the site 4 of TRPV1, wherein the TRPV1 ligand is a naturally occurring compound, optionally Cannabis-derived compound, or synthesized compound.

In some embodiments, the ligand-binding site is site 4A of TRPV1. In some embodiments, the ligand-binding site is a binding pocket of a set of amino acid residues, wherein the amino acid residues comprise: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues (see Table 2). In some embodiments, the ligand-binding site comprises a subset of the amino acid residues comprising: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues (see Table 2). In some embodiments, the ligand-binding site comprises 2, 3, 4, 5, 6, 7, 8, 9, 10, 11, 12, 13, or 14 amino acid residues selected from the group consisting of: Arg 491, Asn 437, Tyr 487, Tyr 444, Tyr 441, Phe 488, Val 440, Tyr 555, Thr 708, Thr 704, Phe 0.434, Tyr 554, Glu 513, and Phe 516 of rat TRPV1 or the closely equivalent human TRPV1 residues (see Table 2).

In some embodiments, the allosteric modulator or the TRPV1 ligand does not initiate TRPV1 dilation and state transition. In some embodiments, neither the allosteric modulator nor the TRPV1 ligand initiates TRPV1 dilation and state transition.

In some embodiments, the allosteric modulator is a terpene naturally present in Cannabis. In some embodiments, the allosteric modulator is Myrcene. In some embodiments, the allosteric modulatory is not Myrcene. In some embodiments, the allosteric modulator is selected from the group consisting of β-ocimene, linalool, nerolidol, and bisabolol. In some embodiments, the allosteric modulatory is β-ocimene. In some embodiments, the allosteric modulatory is linalool. In some embodiments, the allosteric modulatory is nerolidol. In some embodiments, the allosteric modulatory is bisabolol.

In some embodiments, the TRPV1 ligand is cannabidiol (CBD). In some embodiments, the TRPV1 ligand is a cannabinoid other than cannabidiol (CBD) naturally present in Cannabis.

The composition optionally comprises at least one cannabinoid and/or at least one terpene other than the allosteric modulator or the TRPV1 ligand. The composition comprises no more than 20 different species of cannabinoid and terpene compounds, and in typical embodiments is substantially free of THC.

In various embodiments, the pharmaceutical composition comprises no more than 19 different species of cannabinoid and terpene compounds, 18 different species, 17 different species, 16 different species, 15 different species, 14 different species, 13 different species, 12 different species, 11 different species, or no more than 10 different species. In certain embodiments, the pharmaceutical composition comprises no more than 9 different species of cannabinoid and terpene compounds, no more than 8 different species, no more than 7 different species, no more than 6 different species, or no more than 5 different species. In some embodiments, the pharmaceutical composition comprises no more than 4 different species of cannabinoid and terpene compounds, no more than 3 different species, or no more than 2 different species. In a select embodiment, the pharmaceutical composition comprises no more than 1 species of cannabinoid and terpene compounds.

In various embodiments, the pharmaceutical composition comprises at least 2 different species of cannabinoid and terpene compounds, at least 3 different species, at least 4 different species, at least 5 different species, at least 6 different species, at least 7 different species, at least 8 different species, at least 9 different species, or at least 10 different species, in each case comprising no more than 20 different species. In some embodiments, the pharmaceutical composition comprises at least 11 different species of cannabinoid and terpene compounds, at least 12 different species, at least 13 different species, at least 14 different species, or at least 15 different species, in each case comprising no more than 20 different species.

In some embodiments, the pharmaceutical composition comprises 20 different species of cannabinoid and terpene compounds, 19 different species, 18 different species, 17 different species, 16 different species, 15 different species, 14 different species, 13 different species, 12 different species, 11 different species, or 10 different species. In various embodiments, the pharmaceutical composition comprises 9, 8, 7, 6, 5, 4, 3, or 2 different species of cannabinoid and terpene compounds.

In various embodiments, an active compound (i.e., an allosteric modulator or a TRPV1 ligand) is present in an amount that is at least 10% (w/w) of the total content of cannabinoids and terpenes in the pharmaceutical composition. In some embodiments, an active compound is present in an amount that is at least 15% (w/w), at least 20% (w/w), at least 25% (w/w), at least 30% (w/w), at least 35% (w/w), at least 40% (w/w), at least 45% (w/w), or at least 50% (w/w) of the total content of cannabinoids and terpenes in the pharmaceutical composition. In certain embodiments, an active compound is present in an amount that is at least 55% (w/w), at least 60% (w/w), at least 65% (w/w), at least 70% (w/w), at least 75% (w/w), at least 80% (w/w), at least 85% (w/w), or at least 90% (w/w) of the total content of cannabinoids and terpenes in the pharmaceutical composition. In particular embodiments, an active compound is present in an amount that is at least 95% (w/w) of the total content of cannabinoids and terpenes in the pharmaceutical composition.

In various embodiments, an active compound is present in the pharmaceutical composition at a concentration of 0.025%-5% (w/v). In some embodiments, an active compound is present in the pharmaceutical composition at a concentration of 0.025%-2.5% (w/v). In some embodiments, an active compound is present in the pharmaceutical composition at a concentration of 0.025%-1% (w/v). In some embodiments, an active compound is present in the pharmaceutical composition at a concentration of 2% (w/v), 3% (w/v), 4% (w/v), 5% (w/v), 6% (w/v), 7% (w/v), 8% (w/v), 9% (w/v), or 10% (w/v).

In typical embodiments, the allosteric modulator and/or the TRPV1 ligand are present in amounts that are effective to increase TRPV1 calcium flux.

### Other Components

In some embodiments, terpenes and cannabinoids collectively constitute less than 100% by weight (wt %) of the active pharmaceutical ingredient in the pharmaceutical composition.

In various such embodiments, terpenes and cannabinoids collectively constitute at least 75% by weight, but less than 100 wt %, of the pharmaceutically active ingredient. In specific embodiments, terpenes and cannabinoids collectively constitute at least 80%, at least 85%, at least 90%, at least 91%, at least 92%, at least 93%, at least 94%, or at least 95% by weight, but less than 100 wt %, of the active ingredient. In some embodiments, terpenes and cannabinoids collectively constitute at least 96%, at least 97%, at least 98%, or at least 99% by weight, but less than 100 wt %, of the active ingredient.

In embodiments in which terpenes and cannabinoids collectively constitute less than 100% by weight (wt %) of the pharmaceutically active ingredient, the active ingredient further comprises compounds other than terpenes and cannabinoids. In typical such embodiments, all other compounds in the active ingredient are extractable from Cannabis sativa. In specific embodiments, all other compounds in the active ingredient are present in an extract made from Cannabis sativa.

In some embodiments, terpenes and cannabinoids collectively constitute less than 100% (w/v) of the pharmaceutically active ingredient.

### Delta-9 Tetrahydrocannabinol (THC) Content

In typical embodiments, the pharmaceutical composition is either completely or substantially free of delta-9 tetrahydrocannabinol (THC), and thus lacks psychoactive effects, which offers certain regulatory and other physiological advantages.

In certain embodiments, the pharmaceutical composition is not substantially free of THC. In certain of these embodiments, the pharmaceutical composition comprises 1-10 percent by weight (wt %) THC. In specific embodiments, the pharmaceutical composition comprises 2-9 wt % THC, 3-8 wt % THC, 4-7 wt % THC. In certain embodiments, the pharmaceutical composition comprises 1, 2, 3, 4, 5, 6, 7, 8, 9 or 10 wt % THC.

### Nanoparticle

In some embodiments, the pharmaceutical composition further comprises a PLGA nanoparticle. The PLGA nanoparticle can be loaded with the active compound provided in this disclosure. Use of nanoparticle for drug delivery has been described in U.S. application Ser. No. 15/549,653, PCT/ES2019/070765, and U.S. application Ser. No. 16/686,069, incorporated by reference in its entirety herein. Methods and compositions described in U.S. application Ser. No. 15/549,653, PCT/ES2019/070765, U.S. application Ser. No. 16/686,069 or other prior art are used in various embodiments of the present disclosure. Nanoparticles can help delivery of the pharmaceutical compositions of the present disclosures, because nanoparticles can remain in the organism during an extended period of time, thus ensuring correct, controlled and sustained drug delivery. In preferred embodiments, poly(lactic-co-glycolic acid) copolymer (PLGA) is used for its high biocompatibility, low toxicity and high control of drug delivery. Other polymers of interest are: gelatins, dextrans, chitosans, lipids, phospholipids, polycyanoacrylates, polyesters, poly(ε-caprolactone) (PCL) (Hudson and Margaritis, 2014; Lai et al., 2014; Lam and Gambari, 2014).

In some embodiments, PEGylated or partially PEGylated nanoparticles are used. In some embodiments, non-PEGylated nanoparticles are used. In some embodiments, the nanoparticles comprise covalently bonded polyethylene glycol (PEG) and biodegradable and biocompatible poly(lactic-co-glycolic acid) copolymer (PLGA). The presence of PEG chains in the nanoparticles can confer greater stability in acidic environments, such as the stomach, prevents the absorption of proteins and prevents opsonization by the macrophages, increasing the time of systemic circulation.

Nanoparticles can be synthesized using various methods known in the art, for example, the method comprising the steps of: a) Dissolving the PEG-PLGA polymer in a solvent b) Dissolving a lipophilic surfactant in the previous solution c) Dissolving the drug in the previous solution d) Dissolving a hydrophilic surfactant in purified water e) Adding the drug-polymer co-solution (a+b+c) to the surfactant solution (d) drop by drop 0 Evaporating the solvent where the drug and the polymer were dissolved g) Washing the nanoparticles with purified water h) Collecting the nanoparticles i) Adding a cryoprotectant j) Conserving the nanoparticles by freezing.

The PEG-PLGA polymer used in step a) and therefore the nanoparticles of the present invention can have a ratio of lactic acid to glycolic acid ranging from 10% lactic acid and 90% glycolic acid to 90% lactic acid and 10% glycolic acid, any proportion therebetween being possible.

In some embodiments, the PLGA nanoparticle comprises PLGA copolymer having a ratio of lactic acid to glycolic acid between about 10-90% lactic acid and about 90-10% glycolic acid. In some embodiments, the ratio of lactic acid to glycolic acid is about 10% lactic acid to about 90% glycolic acid. In some embodiments, the ratio of lactic acid to glycolic acid is about 25% lactic acid to about 75% glycolic acid. In some embodiments, the ratio of lactic acid to glycolic acid is about 50% lactic acid to about 50% glycolic acid. In some embodiments, the ratio of lactic acid to glycolic acid is about 75% lactic acid to about 25% glycolic acid. In some embodiments, the ratio of lactic acid to glycolic acid is about 90% lactic acid to about 10% glycolic acid.

The molecular weight of the PEG included can vary from 2,000 to 20,000 Da. In a preferred preparation it is 2,000 Da. The PEG is covalently bonded to PLGA.

The solvent used to dissolve the polymer in step a) is any that allows the polymer to be dissolved, for example, but not limited to, acetone or acetonitrile.

The polymer:drug ratio used in the synthesis method of the present invention ranges from 99:1, 95:5, 90:10, 85:15, being any combination that is around this interval. In some embodiments, the polymer:active compound (allosteric modulator or TRPV1 ligand) ratio is between 90:10 and 85:15.

### Formulation

The pharmaceutical composition can be in any form appropriate for administration to humans or non-human animals, including a liquid, an oil, an emulsion, a gel, a colloid, an aerosol, or a solid, and can be formulated for administration by any route of administration appropriate for human or veterinary medicine, including enteral and parenteral routes of administration.

### Pharmacological Compositions Adapted for Administration by Inhalation

In various embodiments, the pharmaceutical composition is formulated for administration by inhalation.

In certain embodiments, the pharmaceutical composition is formulated for administration by a vaporizer. In certain embodiments, the pharmaceutical composition is formulated for administration by a nebulizer. In particular embodiments, the nebulizer is a jet nebulizer or an ultrasonic nebulizer. In certain embodiments, the pharmaceutical composition is formulated for administration by an aerosolizer. In certain embodiments, the pharmaceutical composition is formulated for administration by dry powder inhaler.

In some embodiments, unit dosage forms of the pharmaceutical composition described herein are provided that are adapted for administration of the pharmaceutical composition by vaporizer, nebulizer, aerosolizer, or dry powder inhaler. In some embodiments, the dosage form is a vial, an ampule, optionally scored to allow user opening

In various embodiments, the pharmaceutical composition is an aqueous solution, and can be administered as a nasal or pulmonary spray. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering the solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien Ed., Elsevier Publishers, New York, 1985; M. Naef et al. Development and pharmacokinetic characterization of pulmonal and intravenous delta-9-tetrahydrocannabinol (THC) in humans, J. Pharm. Sci. 93, 1176-84 (2004); and in U.S. Pat. Nos. 4,778,810; 6,080,762; 7,052,678; and 8,277,781 (each incorporated herein by reference). Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Mucosal formulations are, in certain embodiments, administered as dry powder formulations e.g., comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5 micron mass median equivalent aerodynamic diameter (MMEAD), commonly about 1 micron MMEAD, and more typically about 2 micron MMEAD. Maximum particle size appropriate for deposition within the nasal passages is often about 10 micron MMEAD, commonly about 8 micron MMEAD, and more typically about 4 micron MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI) which rely on the patient's breath, upon pulmonary or nasal inhalation, to disperse the power into an aerosolized amount. Alternatively, the dry powder may be administered via air assisted devices that use an external power source to disperse the powder into an aerosolized amount, e.g., a piston pump.

### Pharmacological Compositions Adapted for Oral/Buccal/Sublingual Administration

In various embodiments, the pharmaceutical composition is formulated for oral, buccal, or sublingual administration.

Formulations for oral, buccal or sublingual administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject polypeptide therapeutic agent as an active ingredient. Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In solid dosage forms for oral, buccal or sublingual administration (capsules, tablets, pills, dragees, powders, granules, and the like), one or more therapeutic agents may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

### Pharmacological Compositions Adapted for Injection

In certain embodiments, the pharmaceutical composition is formulated for administration by injection.

For intravenous, intramuscular, or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In various embodiments, the pharmaceutical composition is provided in a unit dosage form. The unit dosage form is a vial, ampule, bottle, or pre-filled syringe. In some embodiments, the unit dosage form contains 0.01 mg, 0.1 mg, 0.5 mg, 1 mg, 2.5 mg, 5 mg, 10 mg, 12.5 mg, 25 mg, 50 mg, 75 mg, or 100 mg of the pharmaceutical composition. In some embodiments, the unit dosage form contains 125 mg, 150 mg, 175 mg, or 200 mg of the pharmaceutical composition. In some embodiments, the unit dosage form contains 250 mg of the pharmaceutical composition.

In typical embodiments, the pharmaceutical composition in the unit dosage form is in liquid form. In various embodiments, the unit dosage form contains between 0.1 mL and 50 ml of the pharmaceutical composition. In some embodiments, the unit dosage form contains 1 ml, 2.5 ml, 5 ml, 7.5 ml, 10 ml, 25 ml, or 50 ml of pharmaceutical composition.

In particular embodiments, the unit dosage form is a vial containing 1 ml of the mixtures containing an allosteric modulator of TRPV1 at a concentration of 0.01 mg/ml, 0.1 mg/ml, 0.5 mg/ml, or 1 mg/ml. In some embodiments, the unit dosage form is a vial containing 2 ml of the mixture containing an allosteric modulator of TRPV1 at a concentration of 0.01 mg/ml, 0.1 mg/ml, 0.5 mg/ml, or 1 mg/ml.

In some embodiments, the pharmaceutical composition in the unit dosage form is in solid form, such as a lyophilate, suitable for solubilization.

Unit dosage form embodiments suitable for subcutaneous, intradermal, or intramuscular administration include preloaded syringes, auto-injectors, and autoinject pens, each containing a predetermined amount of the pharmaceutical composition described hereinabove.

In various embodiments, the unit dosage form is a preloaded syringe, comprising a syringe and a predetermined amount of the pharmaceutical composition. In certain preloaded syringe embodiments, the syringe is adapted for subcutaneous administration. In certain embodiments, the syringe is suitable for self-administration. In particular embodiments, the preloaded syringe is a single use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In certain embodiments, the unit dosage form is an autoinject pen. The autoinject pen comprises an autoinject pen containing a pharmaceutical composition as described herein. In some embodiments, the autoinject pen delivers a predetermined volume of pharmaceutical composition. In other embodiments, the autoinject pen is configured to deliver a volume of pharmaceutical composition set by the user.

In various embodiments, the autoinject pen contains about 0.1 mL to about 5.0 mL of the pharmaceutical composition. In specific embodiments, the autoinject pen contains about 0.5 mL of the pharmaceutical composition. In particular embodiments, the autoinject pen contains about 1.0 mL of the pharmaceutical composition. In other embodiments, the autoinject pen contains about 5.0 mL of the pharmaceutical composition.

### Pharmacological Compositions Adapted for Topical Administration

In various embodiments, the pharmaceutical formulation is formulated for topical administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Suitable topical formulations include those in which the complex mixtures containing an allosteric modulator of TRPV1 featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearoylphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). The mixtures containing an allosteric modulator of TRPV1 featured in the invention may be encapsulated within liposomes or may form complexes thereto, in particular to cationic liposomes. Alternatively, the mixtures containing the allosteric modulatory of TRPV1 may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁-C₁₀ alkyl ester (e.g., isopropylmyristate IPM), monoglyceride, diglyceride or pharmaceutically acceptable salt thereof.

### Process for Preparing Active Ingredient

In some embodiments, the pharmaceutically active ingredient is prepared by mixing chemically pure allosteric modulator of TRPV1, optionally a TRPV1 ligand to desired final concentrations. Each of the compositions can independently be chemically synthesized, either by total synthesis or by synthetic modification of an intermediate, purified from a compositional mixture such as a Cannabis sativa extract, or, as in the Examples described below, purchased commercially.

In other embodiments, the pharmaceutically active ingredient is prepared from a starting compositional mixture by adjusting to predetermined desired final concentrations any one or more of an allosteric modulator of TRPV1, TRPV1 ligand, and other compositions. In typical embodiments, the starting compositional mixture is a Cannabis sativa extract. In currently preferred embodiments, the starting compositional mixture is a Cannabis sativa extract and an allosteric modulator of TRPV1 and optionally a TRPV-1 ligand is added to the mixture to achieve predetermined desired final concentrations.

Typically, in such embodiments, the process further comprises the earlier step of determining the concentration of each desired allosteric modulator of TRPV1, and optional TRPV1 ligand in the starting compositional mixture.

In certain of these embodiments, the process further comprises the still earlier step of preparing a Cannabis sativa extract. Methods of preparing Cannabis sativa extracts are described in U.S. Pat. Nos. 6,403,126, 8,895,078, and 9,066,910; Doorenbos et al., Cultivation, extraction, and analysis of Cannabis sativa L., Annals of The New York Academy of Sciences, 191, 3-14 (1971); Fairbairn and Liebmann, The extraction and estimation of the cannabinoids in Cannabis sativa L. and its products, Journal of Pharmacy and Pharmacology, 25, 150-155 (1973); Oroszlan and Verzar-petri, Separation, quantitation and isolation of cannabinoids from Cannabis sativa L. by overpressured layer chromatography, Journal of Chromatography A, 388, 217-224 (1987), the disclosures of which are incorporated herein by reference in their entireties. In particular embodiments, the extraction method is chosen to provide an extract that has a content of an allosteric modulator of TRPV1, and optional TRPV1 ligand that best approximates the predetermined composition of the active ingredient.

In some embodiments, the process further comprises a first step of selecting a Cannabis sativa strain for subsequent development as a therapeutic agent or a source of extracted compounds for therapy.

In certain embodiments, the strain selected has a typical content in the plant as a whole, or in an extractable portion thereof, of an allosteric modulator of TRPV1, and optional TRPV1 ligand that best approximates the predetermined composition of the active ingredient. In certain embodiments, the strain selected is one that is capable of providing an extract that best approximates the predetermined composition of the active ingredient. In specific embodiments, the strain selected has a typical content in the plant, extractable portion thereof, or extract thereof, that best approximates the predetermined weight ratios of desired allosteric modulator of TRPV1, and optional TRPV1 ligand. In specific embodiments, the strain selected has a typical content in the plant, extractable portion thereof, or extract thereof, that requires adjustment in concentration of the fewest number of the desired allosteric modulator of TRPV1, and optional TRPV1 ligand. In specific embodiments, the strain selected has a typical content in the plant, extractable portion thereof, or extract thereof, that requires the least expensive adjustment in concentration of the desired allosteric modulator of TRPV1, and optional TRPV1 ligand.

### Product by Process

In typical embodiments, the pharmaceutically active ingredient is prepared by one of the processes described in the above section.

In embodiments in which the pharmaceutically active ingredient is prepared from a starting compositional mixture by adjusting to predetermined desired final concentrations any one or more of allosteric modulator of TRPV1, optional TRPV1 ligand, and all other compounds in the active ingredient are present within the starting compositional mixture. In some embodiments in which the starting compositional mixture is a Cannabis sativa extract, all compounds in the active ingredient other than an allosteric modulator of TRPV1, and optional TRPV1 ligand are present within the Cannabis sativa extract.

### Dose Ranges, Generally

In vivo and/or in vitro assays may optionally be employed to help identify optimal dosage ranges for use. The precise dose to be employed in the formulation will also depend on the route of administration, and the seriousness of the condition, and should be decided according to the judgment of the practitioner and each subject's circumstances. Effective doses may be extrapolated from dose-response curves derived from in vitro or animal model test systems.

### Unit Dosage Forms

The pharmaceutical compositions may conveniently be presented in unit dosage form.

The unit dosage form will typically be adapted to one or more specific routes of administration of the pharmaceutical composition.

In various embodiments, the unit dosage form is adapted for administration by inhalation. In certain of these embodiments, the unit dosage form is adapted for administration by a vaporizer. In certain of these embodiments, the unit dosage form is adapted for administration by a nebulizer. In certain of these embodiments, the unit dosage form is adapted for administration by an aerosolizer.

In various embodiments, the unit dosage form is adapted for oral administration, for buccal administration, or for sublingual administration.

In some embodiments, the unit dosage form is adapted for intravenous, intramuscular, or subcutaneous administration.

In some embodiments, the unit dosage form is adapted for intrathecal or intracerebroventricular administration.

In some embodiments, the pharmaceutical composition is formulated for topical administration.

The amount of active ingredient which can be combined with a carrier material to produce a single dosage form will generally be that amount of the compound which produces a therapeutic effect.

### EXAMPLES

The following examples are provided by way of illustration and not limitation.

### Example 1—Mixtures Comprising Terpenes, Cannabinoids, and Both Terpenes and Cannabinoids

We prepared a complex mixture of cannabinoids and terpenes, the Strain A Mixture, based upon the actual chemo-profile of a Cannabis sativa cultivar currently used medicinally in Nevada, USA. Strain chemo-profile data was expressed as % mass and mg/g abundance, and these amounts were converted to amounts to be included in the mixture. The actual chemo-profile was modified in the Strain A Mixture by deliberate omission of THC and THCA and omission of certain labile or insoluble components. We also prepared complex mixtures containing subsets of the compounds in the Strain A Mixture: CBMIX, Cannabinoid Mixture and Terpene Mixture.

All mixtures were prepared by mixing individual components as specified below in Table 1. The Table provides percentage ratios of individual components by weight included in each mixture (Ratio, %). It also provides final concentrations of each component applied to the cell culture in the experiments described below (Conc., μg/ml).

TABLE 1

| | Strain A | | | | Cannabinoid | | Terpene | |
|---|---|---|---|---|---|---|---|---|
| | Mixture | | CBMIX | | Mixture | | Mixture | |
| | Ratio | Conc. | Ratio | Conc. | Ratio | Conc. | Ratio | Conc. |
| | (%) | (μg/ml) | (%) | (μg/ml) | (%) | (μg/ml) | (%) | (μg/ml) |
| Cannabidivarin | 7.42 | 5.6 | 8.40 | 5.6 | 14.56 | 5.6 | 0.00 | 0 |
| (CBDV) | | | | | | | | |
| Cannabichromene | 1.92 | 1.45 | 2.17 | 1.45 | 3.77 | 1.45 | 0.00 | 0 |
| (CBC) | | | | | | | | |
| Cannabidiol | 7.29 | 5.5 | 8.25 | 5.5 | 14.30 | 5.5 | 0.00 | 0 |
| (CBD) | | | | | | | | |
| Cannabidiolic Acid | 4.57 | 3.45 | 5.17 | 3.45 | 8.97 | 3.45 | 0.00 | 0 |
| (CBDA) | | | | | | | | |
| Cannabigerol | 3.64 | 2.75 | 4.12 | 2.75 | 7.15 | 2.75 | 0.00 | 0 |
| (CBG) | | | | | | | | |
| Cannabigerolic Acid | 24.52 | 18.5 | 27.74 | 18.5 | 48.11 | 18.5 | 0.00 | 0 |
| CBGA) | | | | | | | | |
| Cannabinol | 1.59 | 1.2 | 1.80 | 1.2 | 3.12 | 1.2 | 0.00 | 0 |
| (CBN) | | | | | | | | |
| alpha-Bisabolol | 2.58 | 1.95 | 2.92 | 1.95 | 0.00 | 0 | 5.27 | 1.95 |
| alpha-Humulene | 6.03 | 4.55 | 6.82 | 4.55 | 0.00 | 0 | 12.30 | 4.55 |
| α-Pinene | 0.66 | 0.5 | 0.75 | 0.5 | 0.00 | 0 | 1.35 | 0.5 |
| β-Caryophyllene | 14.18 | 10.7 | 16.04 | 10.7 | 0.00 | 0 | 28.92 | 10.7 |
| beta-Myrcene | 11.60 | 8.75 | 0.00 | 0 | 0.00 | 0 | 23.65 | 8.75 |
| (+)-beta-Pinene | 1.33 | 1 | 1.50 | 1 | 0.00 | 0 | 2.70 | 1 |
| Camphene | 0.20 | 0.15 | 0.22 | 0.15 | 0.00 | 0 | 0.41 | 0.15 |
| Limonene | 7.09 | 5.35 | 8.02 | 5.35 | 0.00 | 0 | 14.46 | 5.35 |
| Linalool | 2.39 | 1.8 | 2.70 | 1.8 | 0.00 | 0 | 4.86 | 1.8 |
| Nerolidol | 2.85 | 2.15 | 3.22 | 2.15 | 0.00 | 0 | 5.81 | 2.15 |
| Ocimene | 0.13 | 0.1 | 0.15 | 0.1 | 0.00 | 0 | 0.27 | 0.1 |

Individual components were obtained from various vendors—for example, nerolidol from Tokyo Chemical Industry (#N0454), linalool from Tokyo Chemical Industry (#L0048), alpha-pinene from Sigma Aldrich (#P45680), limonene from MP Biomedicals (#155234), phytol from Ultr Scientific (#FLMS-035), cannabidivarin from Sigma Aldrich (#C-140), cannabichromene from Sigma Aldrich (#C-143), cannabidiol from Sigma Aldrich (#C-045), cannabigerol from Sigma Aldrich (#C-141) and cannabinol from Sigma Aldrich (#C-046). Myrcene, manufactured by MP Biomedical, was obtained from VWR, product #M0235. Each component was mixed as specified above in Table 1.

### Example 2—Cell Culture System for Testing TRPV1-Mediated Calcium Response

The HEK293 cell line was stably transfected with the pcDNA6TR (Invitrogen, CA) plasmid (encoding the tetracycline-sensitive TREx repressor protein), and was maintained in DMEM+10% fetal bovine serum (inactivated at 55° C. for 1 h)+2 mM glutamine in humidified 5% CO₂ atmosphere at 37° C. Selection pressure on the TRex 293 cells was maintained by continuous culture in 10 μg/ml Blasticidin (Sigma, St Louis, Mo.).

For production of TRex HEK293 cells with inducible expression of TRPV1, parental cells were electroporated with the rat TRPV1 cDNA in the pcDNA4TO vector and clonal cell lines were selected by limiting dilution in the presence of 400 μg/ml zeocin (Invitrogen, CA). TRPV1 expression was induced using 1 μg/ml tetracycline for 16 h at 37° C. Stable lines were screened for inducible protein expression using anti-FLAG Western blot, and inducible expression was confirmed. Electrophysiological measurements further confirmed the presence and I/v curve ‘signature’ of TRPV1 in these induced cells. Furthermore, capsaicin-specific calcium fluxes provided in FIG. 1 also confirmed expression and specific response of TRPV1 in the cells, because the calcium flux was not detected in HEK wild type cells without a construct encoding TRPV1.

Calcium responses mediated by TRPV1 were tested by calcium assay in the cell culture system. Cells were washed and incubated with 0.2 μM fluo-4 acetoxymethyl ester (“Fluo-4”) for 30 minutes at 37° C. in a standard modified Ringer's solution of the following composition (in mM): NaCl 145, KCl 2.8, CsCl 10, CaCl2 10, MgCl2 2, glucose 10, Hepes.NaOH 10, pH 7.4, 330 mOsm. Cells were transferred to 96-well plates at 50,000 cells/well and stimulated as indicated. Calcium signals were acquired using a Flexstation 3 (Molecular Devices, Sunnydale, USA). Data was analyzed using SoftMax® Pro 5 (Molecular Devices). Where indicated, nominally calcium-free external conditions were achieved by the preparation of 0 mM CaCl₂ Ringer solution containing 1 mM EGTA. Where indicated, capsaicin (10 μM) and ionomycin (500 nM) were used as positive controls to induce calcium responses. Capsazepine (10 μM) was used where indicated to specifically antagonize TRPV1-mediated calcium responses. Where indicated, baseline traces (no stimulation, NS) were subtracted. Where indicated, vehicle alone traces were subtracted. Where indicated, vehicle comprising various diluents matched to corresponding mixtures was used as a negative control.

### Example 3—TRPV1-Mediated Calcium Influx in Response to Strain a Mixture, Cannabinoid Mixture, or Terpene Mixture

TRPV1-mediated calcium influx was tested in response to the Strain A Mixture, Cannabinoid Mixture and Terpene Mixture as described above. Each mixture was applied to the cell culture medium to expose the cells to final concentrations of individual components as provided in Table 1 (“Conc. μg/ml”). For example, the Strain A Mixture was applied to expose the cells to 5.6 μg/ml of cannabidivarin (CBDV), 8.75 μg/ml of myrcene, etc.

FIGS. 2A-2C provide calcium flux data measured as Fluo-4 relative fluorescence unit (Fluo-4 RFU) over time (sec). As provided in FIGS. 2A-2C, significant calcium fluxes were observed in response to application of the Strain A Mixture (FIG. 2A), and the Terpene Mixture (FIG. 2C), but less so in response to application of the Cannabinoid Mixture (FIG. 2B). The calcium fluxes were not detected in the absence of stimuli (“NS”) or in response to application of vehicle (“veh”) (FIGS. 2A-2C).

When wild-type HEK cells without the TRPV1 construct were presented with the same stimulus conditions, calcium fluxes were not observed (FIGS. 5A-5C). These data demonstrate that the calcium influxes in response to the Strain A Mixture, the Cannabinoid Mixture, or the Terpene Mixture are specific to and mediated by TRPV1.

### Example 4—TRPV1-Mediated Calcium Influx in Response to Individual Terpenes

Because the Terpene Mixture was identified in Example 3 to be largely responsible for the TRPV1-agonistic effects of the Strain A Mixture (see FIGS. 2A-2C), TRPV1-mediated calcium influx was tested in response to individual components of the Terpene Mixture. Each component was applied in the cell culture medium, while fluorescence signals were monitored. Fluorescence signals measured over time are presented in FIGS. 3B-3L for individual terpene compounds.

Significant calcium influx was detected in response to some, but not all, of the terpene compounds tested. In particular, significant calcium flux was detected in response to myrcene (FIG. 3D) and nerolidol (FIG. 3I).

When TRPV1-agonistic effects were compared between myrcene alone and the Terpene Mixture, myrcene was seen to contribute significantly to TRPV1-mediated calcium response, but did not account for 100% of the calcium influx signal. As shown in FIG. 4, the Terpene Mixture (solid curve) had more significant effects than myrcene alone (dotted curve). This suggests that some terpenes, including nerolidol, may have additive or synergistic effects on TRPV1 when applied with myrcene.

### Example 5—Activation of TRPV1 by Myrcene

Myrcene's agonistic effects on TRPV1 were further tested under various conditions. First, TRPV1-mediated calcium flux was tested in response to different concentrations of myrcene (3.5 μg/ml, 1.75 μg/ml, 0.875 μg/ml and 0.43 μg/ml). As illustrated in FIGS. 6A-6D, calcium responses to myrcene were dose-dependent, with the largest flux in response to 3.5 μg/ml of myrcene and the smallest flux in response to 0.43 μg/ml of myrcene. The calcium flux was much smaller in the wild-type HEK cell culture (dotted curves in FIGS. 6A-6D), demonstrating that myrcene induces calcium flux through TRPV1 channel.

Myrcene's agonistic effects on TRPV1 was further confirmed by applying a TRPV1 inhibitor, 10 μM of capsazepine, in the cells activated with 3.5 μg/ml myrcene. As provided in FIG. 7A, calcium flux induced by myrcene diminished in response to capsazepine. As shown in FIG. 7B, calcium flux did not change in response to PBS, applied as a control. The data demonstrate that myrcene induces calcium flux by activating TRPV1.

Activation of TRPV1 by myrcene was also tested under calcium-free medium conditions. Under these conditions, low concentrations of myrcene (0.43 μg/ml, 0.875 μg/ml and 1.75 μg/ml) did not cause increase of calcium-mediated fluorescence (see FIGS. 8B, 8C, and 8D), whereas a high concentration of myrcene (3.5 μg/ml) induced such increase (FIG. 8A). This suggests that myrcene induces calcium flux mostly from extracellular buffer at low concentrations, but can induce calcium flux into the cytosol from intracellular stores at high concentrations. Both extracellular and intracellular fluxes rely on TRPV1, since calcium influx was not observed or were only minimal in wild-type HEK cells without TRPV1 (dotted curves in FIGS. 8A-8D).

To confirm and further investigate the activation of TRPV1 by myrcene, channel currents were assessed via patch clamp experiments in single HEK293 cells overexpressing rat TRPV1. HEK293 cells were kept in sodium-based extracellular Ringer's solution containing 140 mM NaCl, 1 mM CaCl₂, 2 mM MgCl₂, 2.8 mM KCl, 11 mM glucose, and 10 mM HEPES-NaOH, pH 7.2 and osmolarity 300 mOsmol. The cells' cytosol was perfused with intracellular patch pipette solution containing 140 mM Cs-glutamate, 8 mM NaCl, 1 mM MgCl₂, 3 mM MgATP, and 10 mM HEPES-CsOH. The standard internal Ca²⁺ concentration was buffered to 180 nM with 4 mM Ca and 10 mM BAPTA. The level of free unbuffered Ca was adjusted using the calculator provided with WebMaxC (http://www.stanford.edu/˜cpatton/webmaxcS.htm). The pH of the final solution was adjusted to pH 7.2 and osmolarity measured at 300 mOsmol.

TRPV1 channels were activated by adding 5 μM, 10 μM, or 150 μM myrcene to the extracellular solution. 1 μM capsaicin was used as a positive control for TRPV1 activation. Rapid extracellular solution application and exchange was performed with the SmartSquirt delivery system (Auto-Mate Scientific, San Francisco). The system includes a ValveLink TTL interface between the electronic valves and the EPC-9 amplifier (HEKA, Lambrecht, Germany). This configuration allows for programmable solution changes via the PatchMaster software (HEKA, Lambrecht, Germany).

Patch-clamp experiments were performed in the whole-cell configuration at 21-25° C. Patch pipettes had resistances of 2-3 MΩ. Data was acquired with PatchMaster software controlling an EPC-9 amplifier. Voltage ramps of 50 ms spanning the voltage range from −100 to 100 mV were delivered from a holding potential of 0 mV at a rate of 0.5 Hz over a period of 500 ms. Voltages were corrected for a liquid junction potential of 10 mV. Currents were filtered at 2.9 kHz and digitized at 100 us intervals. Capacitive currents were determined and corrected before each voltage ramp. The development of currents for a given potential was extracted from individual ramp current records by measuring the current amplitudes at voltages of −80 mV and +80 mV. Data were analyzed with FitMaster (HEKA, Lambrecht, Germany), and IgorPro (WaveMetrics, Lake Oswego, Oreg., USA). Where applicable, statistical errors of averaged data are given as mean±s.e.m.

As shown in FIGS. 18A-18C, myrcene induced a dose-dependent response in individual cells. Inward and outward current development is shown over time. Each data point (DP) corresponds to approximately 1 second. 5 μM (FIG. 18A), 10 μM (FIG. 18B), and 150 μM (FIG. 18C) myrcene induced 0.5-2.2 nA current compared to 4-10 nA current induced by application of 1 μM capsaicin (not shown). Increasing doses of myrcene result in an inwardly rectifying non-selective cation current which inactivated in a manner dependent both on activation current amplitude (FIGS. 18A-18C) and calcium influx (data not shown).

FIG. 19A shows the same experiment as FIG. 18A, but with the addition of capsaicin after the myrcene application. HEK293 cells overexpressing rat TRPV1 were equilibrated in extracellular Ringer's solution containing 1 mM Ca. The extracellular buffer was exchanged for buffer containing 5 μM myrcene at datapoint (DP) 60. The myrcene solution was exchanged for extracellular buffer containing 1 μM capsaicin at DP 120. Inward and outward currents (nA) were measured at each DP. FIG. 19A shows the average inward and outward currents of 6 independent experiments. 5 μM myrcene induced an approximately 0.5 nA inward current over time, while 1 μM capsaicin induced an approximately 9 nA inward current. Both myrcene and capsaicin also induced an outward current at a lower amplitude than the inward current. FIG. 19B shows a magnified view of the myrcene-induced current.

Next, the relationship between the myrcene- and capsaicin-induced current and the experimental voltage was analyzed. Voltage clamps were performed at data points 1, 59, 119, and 179 (FIG. 19A, arrows 1-4 IV), and the IV relationship assessed before and after addition of myrcene and capsaicin (FIGS. 19C-19E). FIG. 19C shows the break-in current (“1 IV” on FIG. 19A) of the cell and the early current development (“2 IV” on FIG. 19A) in the presence of Ringer's solution. FIG. 19D shows the myrcene-induced TRPV1 activation (“3 IV” on FIG. 19A). FIG. 19E shows the capsaicin-induced TRPV1 activation (“4 IV” on FIG. 19A).

Capsaicin is a TRPV1 agonist known to selectively increase Ca²⁺ ion permeability of the TRPV1 channel. The channel's permeation properties have been previously documented in two states. State 1 for this non-selective cation channel (NSCC) is marginal or no selectivity for calcium over sodium. State 2 (the dilated or transition state) represents an attained state where pore properties have changed to permeate large cations (for example NMDG) and support correspondingly large fluxes of calcium and sodium. The transition from State 1 to State 2 is characterized by a marked linearization of the IV curve with correspondingly larger inward currents than in State 1. FIGS. 18A-18C and FIGS. 19A-19E show that myrcene is a strong activator of TRPV1, producing nA currents. In contrast to capsaicin, myrcene activates the channel primarily in State 1. The differences between the myrcene-induced and capsaicin-induced TRPV1 activation properties suggest that the amplitude, selectivity and therefore physiological outcomes of TRPV1 activation can be manipulated in a rational manner based on differential electrophysiological characteristics of TRPV1-mediated responses to myrcene as opposed to the conventional ligand capsaicin.

### Example 6—Effects of Non-Myrcene Components of the Strain a Mixture on TRPV1

Since myrcene alone could not explain all the TRPV1 agonistic effects of the Strain A Mixture, effects of individual cannabinoids and CBMIX (i.e., the Strain A Mixtures not including myrcene) on TRPV1 were further studied to understand the remaining TRPV1-agonistic effects of the Strain A Mixture.

First, individual cannabinoids were applied in the cell culture medium, while fluorescence signals were monitored. FIGS. 9A-9G illustrate that cannabinoids differentially contribute to calcium fluxes via TRPV1. Modest calcium responses were detected in response to some, but not all, cannabinoid compounds. In particular, calcium flux was detected in response to cannabidivarin (CBDV), cannabichromene (CBC), cannabidiol (CBD), cannabidiolic acid (CBDA), and cannabigerolic acid (CBGA). Such calcium responses were only minimal or absent in cells without TRPV1, demonstrating that the calcium response is mediated by TRPV1.

### Example 7—Network Pharmacology Platform

In order to assess whether myrcene and nerolidol, the two terpenes in our original Cannabis Strain A Mixture with significant TRPV1 agonist effects, had effects at other TRP channels, we developed an in silico prediction approach, termed the GB Sciences Network Pharmacology Platform.

Node and edge data were pulled from the http://bionet.ncpsb.org/batman-tcm/result page source. The data contain source and target information as well as group assignments used to generate Cytoscape network graphs on the website. The node and edge text files were loaded into the R statistical analysis program as comma separated files (csv).

Files were cleaned of superfluous labeling and special characters and then arranged into node and edge data frames with clearly defined variable columns and observation rows using the dplyr library. Node data was reassigned group designations as per the Batman assignments and sorted in alpha order also using the dplyr library. The edge data frame was used to generate a directed network data object using the network library. The network object has two variables added to it: (i) the sorted group assignments from the node data frame, and (ii) the Freeman degree attribute which is calculated from the edge list and assigned to each node using the sna library. Network graphing was rendered through graphing interpreters from the ggnetwork and ggrepel libraries, which render graphs from the network object and use formatting arguments for style.

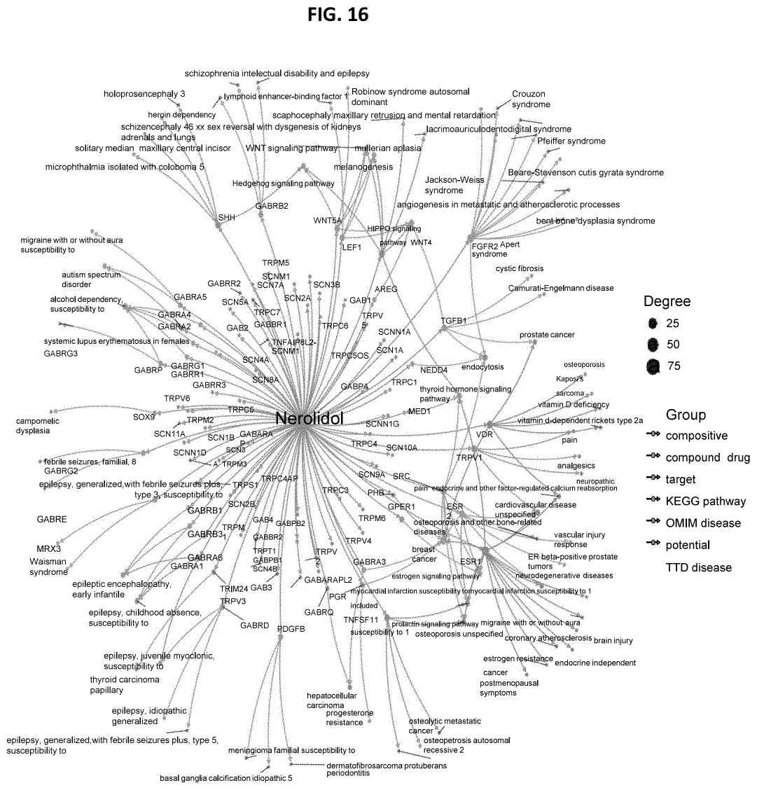

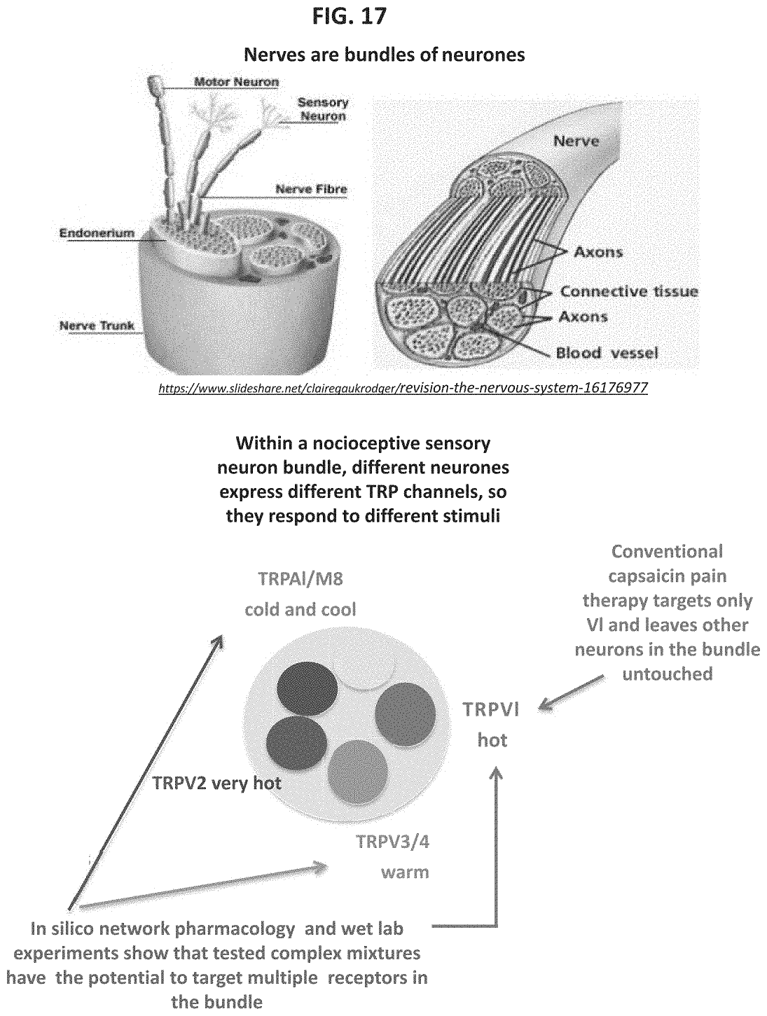

FIG. 15 shows the target analysis and disease-prediction network for myrcene. The presence of multiple TRP channels in the network indicates that efficacy of myrcene will likely extend beyond TRPV1 to other nociceptive neurons in which the primary pain-conducting channel is a distinct TRP. FIG. 16 shows the target analysis and disease-prediction network for nerolidol. The presence of multiple TRP channels in the network indicates that efficacy of nerolidol does not significantly add TRP channels for which myrcene is not indicated.

FIG. 10 illustrates that Therapeutic Target Database (TD) enrichment analysis tends to prioritize myrcene over nerolidol for development in pain and cardiovascular indications. In addition, myrcene contributes significantly to the predicted disease target set for native Cannabis.

FIG. 11 illustrates that diverse ion channel targets are predicted for direct or indirect modulation by myrcene.

### Example 8—Myrcene's Effects on Subsequent TRPV1 Ligand Application

This example demonstrates how Myrcene pre-application and residency at TRPV1 impact subsequent responses of other TRPV1 ligands, such as Cannabidiol (CBD).

First, we confirmed the activity of the second ligand to be used in this allosteric modulation experiment, Cannabidiol.-Cannabidiol (CBD) is an effective ligand for TRPV1, and FIGS. 20A-20D exemplify the CBD-mediated effects on TRPV1 including activation of a current that develops to Imax of up to 5 nA (FIG. 20A), is sensitive to both capsazepine and washout (FIGS. 20B and 20C) and is a rectifying current with Eᵣₑᵥ of ˜0 mV (FIG. 20D).

The potential for Myrcene application to modulate subsequent CBD effects was also explored. As such, Myrcene is initially allowed to saturate the channel under 0 mM external Ca²⁺ concentration, which prohibits influx of Ca²⁺. Cannabidiol is then introduced as a second stimulus to the saturated TRPV1 receptor under 1 mM external Ca²⁺ concentration. The response caused by the second stimulus, Cannabidiol, under such conditions is suppressed as compared Cannabidiol stimulation without prior Myrcene saturation of the TRPV1 receptor as shown in FIG. 21.

These data suggest that Myrcene has the potential to act as an allosteric modulator of other TRPV1 ligands, and prompted an effort to model the interaction sites at TRPV1 for both Cannabidiol and Myrcene, as illustrated below.

### Example 9—Molecular Docking of Myrcene and Cannabidiol at TRPV1

Molecular docking analyses were performed using the Cryo-EM structure of rTRPV1 (RCSB PDB No. 5IS0) to assess potential sites and mechanisms for Myrcene binding. An unbiased computational modeling analysis of the binding of Myrcene in TRPV1 receptor as compared to the binding of Allicin in the TRPV1 receptor was performed (MOE Site Finder, Molecular Operating Environment version 2018, Chemical Computing Group, Montreal, QC). On the basis of its structure, Myrcene is unlikely to participate in the electrophilic additions but is more likely to participate through lipophilic interactions with the channel. Through hydrophobic interactions, over 80 potential binding sites for Myrcene were identified in TRPV1 with many in the region of Cys 621 but no strong interactions with Cys 616 or 621 were observed. Site #4 showed binding of both Allicin and Myrcene, but a lower docking energy (−17.7 kcal/mol) for Myrcene was observed over that of Allicin (−14.0 kcal/mol), despite the fact that Allicin was able to interact with the Cysteines, likely in a covalent manner, whereas Myrcene was only able to interact through hydrophobic interactions primarily with Arg 491 and Tyr 554 but also with other residues, such as F488, N437, F434, Y555, 5512, E513 and F516, as shown in FIGS. 22A and 22B. Each of these residues is identical or closely conserved between rat or human TRPV1 and most have been implicated previously as of importance in ligand binding or regulation of TRPV1. For example, prior studies showed a Tyr 554 to alanine mutation ablated both capsaicin and resiniferatoxin binding in TRPV1. These relationships are fully described in Table 2 and include a protonation site (R491), capsaicin interacting sites and residues that contribute to the hydrophobic interior of transmembrane domains S1, 2, and 4. Several sites were also involved in voltage or thermal sensing, and based on mutagenesis studies, this Myrcene binding site, site #4, might also be sensitive to Resiniferatoxin competition. Residues close to the S4-S5 linker, a key regulatory region for TRPs, were also implicated in the binding of Myrcene.

Table 2 shows an analysis of the residues implicated in binding Myrcene and Cannabidiol in TRPV1 by our molecular docking analysis. The implicated residues are shown at left. The second column identifies whether the residues is in the S4-S5 linker. The third column identifies whether the residue is exactly conserved in human and which human residue is equivalent if not completely conserved. The third and fourth columns comprise a literature review of these residues, summarizing prior studies as to their role and effect of any mutagenesis that have been carried out. The fourth column provides the literature reference for the cited studies in the form of a Pub Med ID (PMID).

TABLE 2

| | | | |
|---|---|---|---|
| S4-S5 | Conserved | | |
| linker | in Human | Role/effect of | Reference |
| (AA554-582?) | (UniProt)? | mutagenesis | (PMID) |

MYR binding pocket residues (Rat CRYO-EM STRUCTURE site 4)

| | | | | |
|---|---|---|---|---|
| F488 | | Yes | Contributes to | 24305160 |
| | | | hydrophobic interior | |
| | | | of S1, 3, 4 | |
| F491 | | Yes | ↓ Capsaicin sensitivity, | 11853675 |
| | | | protonation site | |
| F516 | | Yes | Contributes to | 24305160 |
| | | | hydrophobic interior | |
| | | | of S1,3,4 | |
| N437 | | Yes | Capsaicin interaction | 25809255 |
| F434 | | ~F435 | Capsaicin interaction | 25809255 |
| Y555 | Yes | Yes | ↓ voltage sensing, | 21044960 |
| | | | capsaicin insensitivity | |
| Y554 | Yes | Yes | ↓ voltage sensing, | 21044960 |
| | | | capsaicin insensitivity | |
| S512 | | Yes | ↓ RTX binding | 27335334 |
| | | | | 24305161 |
| | | | | 23800232 |
| E513 | | Yes | Some effect on | 23800232 |
| | | | thermal/pH threshold | 11853675 |

CBD binding pocket residues (Rat CRYO-EM STRUCTURE site 4A)

| | | | | |
|---|---|---|---|---|
| F434 | | ~F435 | Capsaicin interaction | 25809255 |
| T704 | | ~T705 | ↓ activation by PMA, | 14523239 |
| | | | ↓ vanilloid binding, | |
| | | | capsaicin insensitivity | |
| T708 | | ~T708 | | |
| Y555 | Yes | Yes | ↓ voltage sensing, | 21044960 |
| | | | capsaicin insensitivity | |
| V440 | | ~V441 | | |
| F488 | | Yes | Contributes to | 24305160 |
| | | | hydrophobic interior | |
| | | | of S1, 3, 4 | |
| Y441 | | ~Y442 | Ligand insensitivity | 21044960 |
| Y444 | | ~Y445 | | 23220012 |
| Y487 | | Yes | Implicated in Camphor | 22314297 |
| | | | binding to TRPV1 | |
| N437 | | ~N438 | Implicated in | 25809255 |
| | | | Capsaicin interaction | |
| R491 | | Yes | ↓ Capsaicin sensitivity, | 11853675 |
| | | | protonation site | |
| F516 | | Yes | Contributes to | 24305160 |
| | | | hydrophobic interior | |
| | | | of S1, 3, 4 | |
| E513 | | Yes | Some effect on | 23800232 |
| | | | thermal/pH threshold | 11853675 |
| Y554 | Yes | Yes | ↓ voltage sensing, | 21044960 |
| | | | capsaicin insensitivity | |

One chemical moiety in Myrcene that is contacted by several residues in the binding site is a dimethyl group that is shared by many other terpenes found in Cannabis, such as Ocimene, Linalool, Nerolidol, and Bisabolol, and other plant sources as illustrated in FIG. 23, and may also have the capacity to occupy this binding site. Interestingly, we have shown that Nerolidol also activated TRPV1-mediated Ca²⁺ influx, as shown in FIG. 3I, but similar fluxes with the other compounds at the same doses were not observed. Given that other terpenes have different structures than the ones discussed (e.g., humulene, not shown), these data offer a pre-screen approach for decisions as to which of the large number of terpene molecules should be prioritized for exploration in the context of TRPV1 and nociception.

The CBD binding site was also investigated similarly, as shown in FIGS. 25A, 25B and 26. A binding pocket that partially overlaps with that of Myrcene was identified wherein the docking of CBD was calculated to be −26.5 kcal/mol. In this site, Y554 and R491 appear to provide key interactions with CBD, similar to the case for Myrcene. The remaining residues implicated in CBD binding show both similarities and differences to the Myrcene site. The residues implicated in the bindings of Myrcene and CBD across a two-dimensional representation of the channel's protein sequence is shown in FIGS. 26 and 27. Furthermore, Table 2 tabulates each implicated residue, its conservation or identity between rat and human, location to the S4-5 linker, function, effects of mutagenesis where known, and supporting references.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A method of designing a complex mixture for treating pain through targeting a TRP channel selected from TRPV1, TRPV2, TRPM8 and TRPA1, comprising the steps of:
analyzing compounds in Cannabis or other plants using in vitro or in silico technique and predicting whether each of the compounds binds to site 4 or site 4A of TRPV1, thereby differentiating between likely analgesic and non-analgesic compounds;
selecting a subset of the compounds that contain a functional dimethyl moiety and excluding a different subset of the compounds that do not contain the functional dimethyl moiety, thereby obtaining selected compounds; and
designing the complex mixture comprising the selected compounds.

2. The method of claim 1, wherein the site 4 of TRPV1 is a binding pocket of a set of amino acid residues, wherein the amino acid residues comprise: Arg 491, Asn 437, Phe 434, Tyr 555, Ser 512, Tyr 554, Glu 513, Phe 516, and Phe 488 of rat TRPV1 or the closely equivalent human TRPV1 residues.

3. The method of claim 1, wherein the site 4A of TRPV1 is a binding pocket of a set of amino acid residues, wherein the amino acid residues comprise: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues.

4. The method of claim 1, further comprising the step of identifying compounds that do not initiate state transition or pore dilation in TRPV1.

5. A method of treating pain in a mammalian subject, comprising the steps of:
administering to the subject a pharmaceutical composition, in an amount, by a route of administration, and for a time sufficient to cause TRPV1 inactivation or desensitization in sensory neurons within the subject,
wherein the pharmaceutical composition comprises an active compound capable of activating TRPV1 by binding to site 4 or 4A of TRPV1, and a pharmaceutically acceptable carrier or diluent; and
wherein the active compound is (i) a naturally occurring compound, optionally a Cannabis-derived compound, or (ii) a synthetic compound.

6. (canceled)

7. The method of claim 5, wherein the site 4 of TRPV1 is a binding pocket of a set of amino acid residues, wherein the amino acid residues comprise: Arg 491, Asn 437, Phe 434, Tyr 555, Ser 512, Tyr 554, Glu 513, Phe 516, and Phe 488 of rat TRPV1 or the closely equivalent human TRPV1 residues.

8. The method of claim 5, wherein the site 4A of TRPV1 is a binding pocket of a set of amino acid residues, wherein the amino acid residues comprise: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues.

9. The method of claim 5, wherein the active compound is selected from the group consisting of β-ocimene, linalool, nerolidol, and bisabolol.

10. The method of claim 5, wherein the active compound is Myrcene or Cannabidiol (CBD).

11. (canceled)

12. (canceled)

13. The method of claim 5, wherein the pharmaceutical composition further comprises a PLGA nanoparticle.

14. The method of claim 13, wherein the PLGA nanoparticle comprises PLGA copolymer having a ratio of lactic acid to glycolic acid between about 10-90% lactic acid and about 90-10% glycolic acid.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A method of treating pain in a mammalian subject, comprising the steps of:
administering to the subject a pharmaceutical composition, in an amount, by a route of administration, and for a time sufficient to cause TRPV1 inactivation or desensitization in sensory neurons within the subject,
wherein the pharmaceutical composition comprises (i) an allosteric modulator capable of activating TRPV1 by binding to site 4 of TRPV1, (ii) a TRPV1 ligand capable of activating TRPV1 by binding to a ligand-binding site at least partially overlapping with the site 4 of TRPV1, and (iii) a pharmaceutically acceptable carrier or diluent,
wherein the allosteric modulator and the TRPV1 ligand is naturally occurring, optionally Cannabis-derived, or synthesized; and
wherein the allosteric modulator and the TRPV1 ligand are different compounds.

36. The method of claim 35, wherein the allosteric modulator is Myrcene.

37. (canceled)

38. The method of claim 35, wherein the allosteric modulator is selected from the group consisting of β-ocimene, linalool, nerolidol, and bisabolol.

39. The method of claim 35, wherein the TRPV1 ligand is cannabidiol (CBD).

40. The method of claim 35, wherein the ligand-binding site is site 4A.

41. The method of claim 35, wherein the site 4 of TRPV1 is a binding pocket of a set of amino acid residues, wherein the amino acid residues comprise: Arg 491, Asn 437, Phe 434, Tyr 555, Ser 512, Tyr 554, Glu 513, Phe 516, and Phe 488 of rat TRPV1 or the closely equivalent human TRPV1 residues.

42. The method of claim 40, wherein the site 4A of TRPV1 is a binding pocket of a set of amino acid residues, wherein the amino acid residues comprise: Arg 491, Asn 437, Tyr 487, Tyr 444, Tyr 441, Phe 488, Val 440, Tyr 555, Thr 708, Thr 704, Phe 434, Tyr 554, Glu 513, and Phe 516 of rat TRPV1 or the closely equivalent human TRPV1 residues.

43. (canceled)

44. The method of claim 35, wherein the pharmaceutical composition further comprises a PLGA nanoparticle.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A pharmaceutical composition, comprising:
an allosteric modulator capable of activating TRPV1 by binding to site 4 of TRPV1 and a pharmaceutically acceptable carrier or diluent,
wherein the composition is substantially free from THC; and
wherein the allosteric modulator is a naturally occurring compound, optionally Cannabis-derived compound, or synthesized compound.

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)
